SEQUENCE LISTING (59 sequences)

SEQ ID NO: 1 - protein, 389 aa, Homo sapiens
MLSLRVPLAPITDPQQLQLSPLKGLSLVDKENTPPALSGTRVLASKTARRIFQEPTEPKTKAAAPGVEDEPLLRENPRRFVIFPIEYHDIWQMYKKAEASFWTAEEVDLSKDIQHWESLKPEERYFISHVLAFFAASDGIVNENLVERFSQEVQITEARCFYGFQIAMENIHSEMYSLLIDTYIKDPKEREFLFNAIETMPCVKKKADWALRWIGDKEATYGERVVAFAAVEGIFFSGSFASIFWLKKRGLMPGLTFSNELISRDEGLHCDFACLMFKHLVHKPSEERVREIIINAVRIEQEFLTEALPVKLIGMNCTLMKQYIEFVADRLMLELGFSKVFRVENPFDFMENISLEGKTNFFEKRVGEYQRMGVMSSPTENSFTLDADF

SEQ ID NO: 2 - DNA, 1170 bp, Homo sapiens
  CDS (1)...(1167)
atgctctccctccgtgtcccgctcgcgcccatcacggacccgcagcagctgcagctctcgccgctgaaggggctcagcttggtcgacaaggagaacacgccgccggccctgagcgggacccgcgtcctggccagcaagaccgcgaggaggatcttccaggagcccacggagccgaaaactaaagcagctgcccccggcgtggaggatgagccgctgctgagagaaaacccccgccgctttgtcatcttccccatcgagtaccatgatatctggcagatgtataagaaggcagaggcttccttttggaccgccgaggaggttgacctctccaaggacattcagcactgggaatccctgaaacccgaggagagatattttatatcccatgttctggctttctttgcagcaagcgatggcatagtaaatgaaaacttggtggagcgatttagccaagaagttcagattacagaagcccgctgtttctatggcttccaaattgccatggaaaacatacattctgaaatgtatagtcttcttattgacacttacataaaagatcccaaagaaagggaatttctcttcaatgccattgaaacgatgccttgtgtcaagaagaaggcagactgggccttgcgctggattggggacaaagaggctacctatggtgaacgtgttgtagcctttgctgcagtggaaggcattttcttttccggttcttttgcgtcgatattctggctcaagaaacgaggactgatgcctggcctcacattttctaatgaacttattagcagagatgagggtttacactgtgattttgcttgcctgatgttcaaacacctggtacacaaaccatcggaggagagagtaagagaaataattatcaatgctgttcggatagaacaggagttcctcactgaggccttgcctgtgaagctcattgggatgaattgcactctaatgaagcaatacattgagtttgtggcagacagacttatgctggaactgggttttagcaaggttttcagagtagagaacccatttgactttatggagaatatttcactggaaggaaagactaacttctttgagaagagagtaggcgagtatcagaggatgggagtgatgtcaagtccaacagagaattcttttaccttggatgctgacttctaa

SEQ ID NO: 3 - protein, 389 aa, Homo sapiens
MLSLRVPLAPITDPQQLQLSPLKGLSLVDKENTPPALSGTRVLASKTARRIFQEPTEPKTKAAAPGVEDEPLLRENPRRFVIFPIEYHDIWQMYKKAEASFWTAEEVDLSKDIQHWESLKPEERYFISHVLAFFAASDGIVNENLVERFSQEVQITEARCFFGFQIAMENIHSEMYSLLIDTYIKDPKEREFLFNAIETMPCVKKKADWALRWIGDKEATYGERVVAFAAVEGIFFSGSFASIFWLKKRGLMPGLTFSNELISRDEGLHCDFACLMFKHLVHKPSEERVREIIINAVRIEQEFLTEALPVKLIGMNCTLMKQYIEFVADRLMLELGFSKVFRVENPFDFMENISLEGKTNFFEKRVGEYQRMGVMSSPTENSFTLDADF

SEQ ID NO: 4 - DNA, 1170 bp, Homo sapiens
atgctctccctccgtgtcccgctcgcgcccatcacggacccgcagcagctgcagctctcgccgctgaaggggctcagcttggtcgacaaggagaacacgccgccggccctgagcgggacccgcgtcctggccagcaagaccgcgaggaggatcttccaggagcccacggagccgaaaactaaagcagctgcccccggcgtggaggatgagccgctgctgagagaaaacccccgccgctttgtcatcttccccatcgagtaccatgatatctggcagatgtataagaaggcagaggcttccttttggaccgccgaggaggttgacctctccaaggacattcagcactgggaatccctgaaacccgaggagagatattttatatcccatgttctggctttctttgcagcaagcgatggcatagtaaatgaaaacttggtggagcgatttagccaagaagttcagattacagaagcccgctgtttctttggcttccaaattgccatggaaaacatacattctgaaatgtatagtcttcttattgacacttacataaaagatcccaaagaaagggaatttctcttcaatgccattgaaacgatgccttgtgtcaagaagaaggcagactgggccttgcgctggattggggacaaagaggctacctatggtgaacgtgttgtagcctttgctgcagtggaaggcattttcttttccggttcttttgcgtcgatattctggctcaagaaacgaggactgatgcctggcctcacattttctaatgaacttattagcagagatgagggtttacactgtgattttgcttgcctgatgttcaaacacctggtacacaaaccatcggaggagagagtaagagaaataattatcaatgctgttcggatagaacaggagttcctcactgaggccttgcctgtgaagctcattgggatgaattgcactctaatgaagcaatacattgagtttgtggcagacagacttatgctggaactgggttttagcaaggttttcagagtagagaacccatttgactttatggagaatatttcactggaaggaaagactaacttctttgagaagagagtaggcgagtatcagaggatgggagtgatgtcaagtccaacagagaattcttttaccttggatgctgacttctaa

SEQ ID NO: 5 - protein, 389 aa, Homo sapiens
MLSLRVPLAPITDPQQLQLSPLKGLSLVDKENTPPALSGTRVLASKTARRIFQEPTEPKTKAAAPGVEDEPLLRENPRRFVIFPIEYHDIWQMYKKAEASFWTAEEVDLSKDIQHWESLKPEERYFISHVLAFFAASDGIVNENLVERFSQEVQITEARCFWGFQIAMENIHSEMYSLLIDTYIKDPKEREFLFNAIETMPCVKKKADWALRWIGDKEATYGERVVAFAAVEGIFFSGSFASIFWLKKRGLMPGLTFSNELISRDEGLHCDFACLMFKHLVHKPSEERVREIIINAVRIEQEFLTEALPVKLIGMNCTLMKQYIEFVADRLMLELGFSKVFRVENPFDFMENISLEGKTNFFEKRVGEYQRMGVMSSPTENSFTLDADF

SEQ ID NO: 6 - DNA, 1170 bp, Homo sapiens
atgctctccctccgtgtcccgctcgcgcccatcacggacccgcagcagctgcagctctcgccgctgaaggggctcagcttggtcgacaaggagaacacgccgccggccctgagcgggacccgcgtcctggccagcaagaccgcgaggaggatcttccaggagcccacggagccgaaaactaaagcagctgcccccggcgtggaggatgagccgctgctgagagaaaacccccgccgctttgtcatcttccccatcgagtaccatgatatctggcagatgtataagaaggcagaggcttccttttggaccgccgaggaggttgacctctccaaggacattcagcactgggaatccctgaaacccgaggagagatattttatatcccatgttctggctttctttgcagcaagcgatggcatagtaaatgaaaacttggtggagcgatttagccaagaagttcagattacagaagcccgctgtttctggggcttccaaattgccatggaaaacatacattctgaaatgtatagtcttcttattgacacttacataaaagatcccaaagaaagggaatttctcttcaatgccattgaaacgatgccttgtgtcaagaagaaggcagactgggccttgcgctggattggggacaaagaggctacctatggtgaacgtgttgtagcctttgctgcagtggaaggcattttcttttccggttcttttgcgtcgatattctggctcaagaaacgaggactgatgcctggcctcacattttctaatgaacttattagcagagatgagggtttacactgtgattttgcttgcctgatgttcaaacacctggtacacaaaccatcggaggagagagtaagagaaataattatcaatgctgttcggatagaacaggagttcctcactgaggccttgcctgtgaagctcattgggatgaattgcactctaatgaagcaatacattgagtttgtggcagacagacttatgctggaactgggttttagcaaggttttcagagtagagaacccatttgactttatggagaatatttcactggaaggaaagactaacttctttgagaagagagtaggcgagtatcagaggatgggagtgatgtcaagtccaacagagaattcttttaccttggatgctgacttctaa

SEQ ID NO: 7 - protein, 389 aa, Homo sapiens
MLSLRVPLAPITDPQQLQLSPLKGLSLVDKENTPPALSGTRVLASKTARRIFQEPTEPKTKAAAPGVEDEPLLRENPRRFVIFPIEYHDIWQMYKKAEASFWTAEEVDLSKDIQHWESLKPEERYFISHVLAFFAASDGIVNENLVERFSQEVQITEARCFFGFQIAMENIHSEMFSLLIDTYIKDPKEREFLFNAIETMPCVKKKADWALRWIGDKEATYGERVVAFAAVEGIFFSGSFASIFWLKKRGLMPGLTFSNELISRDEGLHCDFACLMFKHLVHKPSEERVREIIINAVRIEQEFLTEALPVKLIGMNCTLMKQYIEFVADRLMLELGFSKVFRVENPFDFMENISLEGKTNFFEKRVGEYQRMGVMSSPTENSFTLDADF

SEQ ID NO: 8 - DNA, 1170 bp, Homo sapiens
atgctctccctccgtgtcccgctcgcgcccatcacggacccgcagcagctgcagctctcgccgctgaaggggctcagcttggtcgacaaggagaacacgccgccggccctgagcgggacccgcgtcctggccagcaagaccgcgaggaggatcttccaggagcccacggagccgaaaactaaagcagctgcccccggcgtggaggatgagccgctgctgagagaaaacccccgccgctttgtcatcttccccatcgagtaccatgatatctggcagatgtataagaaggcagaggcttccttttggaccgccgaggaggttgacctctccaaggacattcagcactgggaatccctgaaacccgaggagagatattttatatcccatgttctggctttctttgcagcaagcgatggcatagtaaatgaaaacttggtggagcgatttagccaagaagttcagattacagaagcccgctgtttctttggcttccaaattgccatggaaaacatacattctgaaatgtttagtcttcttattgacacttacataaaagatcccaaagaaagggaatttctcttcaatgccattgaaacgatgccttgtgtcaagaagaaggcagactgggccttgcgctggattggggacaaagaggctacctatggtgaacgtgttgtagcctttgctgcagtggaaggcattttcttttccggttcttttgcgtcgatattctggctcaagaaacgaggactgatgcctggcctcacattttctaatgaacttattagcagagatgagggtttacactgtgattttgcttgcctgatgttcaaacacctggtacacaaaccatcggaggagagagtaagagaaataattatcaatgctgttcggatagaacaggagttcctcactgaggccttgcctgtgaagctcattgggatgaattgcactctaatgaagcaatacattgagtttgtggcagacagacttatgctggaactgggttttagcaaggttttcagagtagagaacccatttgactttatggagaatatttcactggaaggaaagactaacttctttgagaagagagtaggcgagtatcagaggatgggagtgatgtcaagtccaacagagaattcttttaccttggatgctgacttctaa

SEQ ID NO: 9 - protein, 389 aa, Homo sapiens
MLSLRVPLAPITDPQQLQLSPLKGLSLVDKENTPPALSGTRVLASKTARRIFQEPTEPKTKAAAPGVEDEPLLRENPRRFVIFPIEYHDIWQMYKKAEASFWTAEEVDLSKDIQHWESLKPEERYFISHVLAFFAASDGIVNENLVERFSQEVQITEARCFFGFQIAMENIHSEMWSLLIDTYIKDPKEREFLFNAIETMPCVKKKADWALRWIGDKEATYGERVVAFAAVEGIFFSGSFASIFWLKKRGLMPGLTFSNELISRDEGLHCDFACLMFKHLVHKPSEERVREIIINAVRIEQEFLTEALPVKLIGMNCTLMKQYIEFVADRLMLELGFSKVFRVENPFDFMENISLEGKTNFFEKRVGEYQRMGVMSSPTENSFTLDADF

SEQ ID NO: 10 - DNA, 1170 bp, Homo sapiens
atgctctccctccgtgtcccgctcgcgcccatcacggacccgcagcagctgcagctctcgccgctgaaggggctcagcttggtcgacaaggagaacacgccgccggccctgagcgggacccgcgtcctggccagcaagaccgcgaggaggatcttccaggagcccacggagccgaaaactaaagcagctgcccccggcgtggaggatgagccgctgctgagagaaaacccccgccgctttgtcatcttccccatcgagtaccatgatatctggcagatgtataagaaggcagaggcttccttttggaccgccgaggaggttgacctctccaaggacattcagcactgggaatccctgaaacccgaggagagatattttatatcccatgttctggctttctttgcagcaagcgatggcatagtaaatgaaaacttggtggagcgatttagccaagaagttcagattacagaagcccgctgtttctttggcttccaaattgccatggaaaacatacattctgaaatgtggagtcttcttattgacacttacataaaagatcccaaagaaagggaatttctcttcaatgccattgaaacgatgccttgtgtcaagaagaaggcagactgggccttgcgctggattggggacaaagaggctacctatggtgaacgtgttgtagcctttgctgcagtggaaggcattttcttttccggttcttttgcgtcgatattctggctcaagaaacgaggactgatgcctggcctcacattttctaatgaacttattagcagagatgagggtttacactgtgattttgcttgcctgatgttcaaacacctggtacacaaaccatcggaggagagagtaagagaaataattatcaatgctgttcggatagaacaggagttcctcactgaggccttgcctgtgaagctcattgggatgaattgcactctaatgaagcaatacattgagtttgtggcagacagacttatgctggaactgggttttagcaaggttttcagagtagagaacccatttgactttatggagaatatttcactggaaggaaagactaacttctttgagaagagagtaggcgagtatcagaggatgggagtgatgtcaagtccaacagagaattcttttaccttggatgctgacttctaa

SEQ ID NO: 11 - protein, 389 aa, Homo sapiens
MLSLRVPLAPITDPQQLQLSPLKGLSLVDKENTPPALSGTRVLASKTARRIFQEPTEPKTKAAAPGVEDEPLLRENPRRFVIFPIEYHDIWQMYKKAEASFWTAEEVDLSKDIQHWESLKPEERYFISHVLAFFAASDGIVNENLVERFSQEVQITEARCFWGFQIAMENIHSEMFSLLIDTYIKDPKEREFLFNAIETMPCVKKKADWALRWIGDKEATYGERVVAFAAVEGIFFSGSFASIFWLKKRGLMPGLTFSNELISRDEGLHCDFACLMFKHLVHKPSEERVREIIINAVRIEQEFLTEALPVKLIGMNCTLMKQYIEFVADRLMLELGFSKVFRVENPFDFMENISLEGKTNFFEKRVGEYQRMGVMSSPTENSFTLDADF

SEQ ID NO: 12 - DNA, 1170 bp, Homo sapiens
atgctctccctccgtgtcccgctcgcgcccatcacggacccgcagcagctgcagctctcgccgctgaaggggctcagcttggtcgacaaggagaacacgccgccggccctgagcgggacccgcgtcctggccagcaagaccgcgaggaggatcttccaggagcccacggagccgaaaactaaagcagctgcccccggcgtggaggatgagccgctgctgagagaaaacccccgccgctttgtcatcttccccatcgagtaccatgatatctggcagatgtataagaaggcagaggcttccttttggaccgccgaggaggttgacctctccaaggacattcagcactgggaatccctgaaacccgaggagagatattttatatcccatgttctggctttctttgcagcaagcgatggcatagtaaatgaaaacttggtggagcgatttagccaagaagttcagattacagaagcccgctgtttctggggcttccaaattgccatggaaaacatacattctgaaatgtttagtcttcttattgacacttacataaaagatcccaaagaaagggaatttctcttcaatgccattgaaacgatgccttgtgtcaagaagaaggcagactgggccttgcgctggattggggacaaagaggctacctatggtgaacgtgttgtagcctttgctgcagtggaaggcattttcttttccggttcttttgcgtcgatattctggctcaagaaacgaggactgatgcctggcctcacattttctaatgaacttattagcagagatgagggtttacactgtgattttgcttgcctgatgttcaaacacctggtacacaaaccatcggaggagagagtaagagaaataattatcaatgctgttcggatagaacaggagttcctcactgaggccttgcctgtgaagctcattgggatgaattgcactctaatgaagcaatacattgagtttgtggcagacagacttatgctggaactgggttttagcaaggttttcagagtagagaacccatttgactttatggagaatatttcactggaaggaaagactaacttctttgagaagagagtaggcgagtatcagaggatgggagtgatgtcaagtccaacagagaattcttttaccttggatgctgacttctaa

SEQ ID NO: 13 - protein, 389 aa, Homo sapiens
MLSLRVPLAPITDPQQLQLSPLKGLSLVDKENTPPALSGTRVLASKTARRIFQEPTEPKTKAAAPGVEDEPLLRENPRRFVIFPIEYHDIWQMYKKAEASFWTAEEVDLSKDIQHWESLKPEERYFISHVLAFFAASDGIVNENLVERFSQEVQITEARCFWGFQIAMENIHSEMWSLLIDTYIKDPKEREFLFNAIETMPCVKKKADWALRWIGDKEATYGERVVAFAAVEGIFFSGSFASIFWLKKRGLMPGLTFSNELISRDEGLHCDFACLMFKHLVHKPSEERVREIIINAVRIEQEFLTEALPVKLIGMNCTLMKQYIEFVADRLMLELGFSKVFRVENPFDFMENISLEGKTNFFEKRVGEYQRMGVMSSPTENSFTLDADF

SEQ ID NO: 14 - DNA, 1170 bp, Homo sapiens
atgctctccctccgtgtcccgctcgcgcccatcacggacccgcagcagctgcagctctcgccgctgaaggggctcagcttggtcgacaaggagaacacgccgccggccctgagcgggacccgcgtcctggccagcaagaccgcgaggaggatcttccaggagcccacggagccgaaaactaaagcagctgcccccggcgtggaggatgagccgctgctgagagaaaacccccgccgctttgtcatcttccccatcgagtaccatgatatctggcagatgtataagaaggcagaggcttccttttggaccgccgaggaggttgacctctccaaggacattcagcactgggaatccctgaaacccgaggagagatattttatatcccatgttctggctttctttgcagcaagcgatggcatagtaaatgaaaacttggtggagcgatttagccaagaagttcagattacagaagcccgctgtttctggggcttccaaattgccatggaaaacatacattctgaaatgtggagtcttcttattgacacttacataaaagatcccaaagaaagggaatttctcttcaatgccattgaaacgatgccttgtgtcaagaagaaggcagactgggccttgcgctggattggggacaaagaggctacctatggtgaacgtgttgtagcctttgctgcagtggaaggcattttcttttccggttcttttgcgtcgatattctggctcaagaaacgaggactgatgcctggcctcacattttctaatgaacttattagcagagatgagggtttacactgtgattttgcttgcctgatgttcaaacacctggtacacaaaccatcggaggagagagtaagagaaataattatcaatgctgttcggatagaacaggagttcctcactgaggccttgcctgtgaagctcattgggatgaattgcactctaatgaagcaatacattgagtttgtggcagacagacttatgctggaactgggttttagcaaggttttcagagtagagaacccatttgactttatggagaatatttcactggaaggaaagactaacttctttgagaagagagtaggcgagtatcagaggatgggagtgatgtcaagtccaacagagaattcttttaccttggatgctgacttctaa

SEQ ID NO: 15 - protein, 351 aa, Homo sapiens
MGDPERPEAAGLDQDERSSSDTNESEIKSNEEPLLRKSSRRFVIFPIQYPDIWKMYKQAQASFWTAEEVDLSKDLPHWNKLKADEKYFISHILAFFAASDGIVNENLVERFSQEVQVPEARCFYGFQILIENVHSEMYSLLIDTYIRDPKKREFLFNAIETMPYVKKKADWALRWIADRKSTFGERVVAFAAVEGVFFSGSFAAIFWLKKRGLMPGLTFSNELISRDEGLHCDFACLMFQYLVNKPSEERVREIIVDAVKIEQEFLTEALPVGLIGMNCILMKQYIEFVADRLLVELGFSKVFQAENPFDFMENISLEGKTNFFEKRVSEYQRFAVMAETTDNVFTLDADF

SEQ ID NO: 16 - DNA, 1056 bp, Homo sapiens
  CDS (1)...(1056)
atgggcgacccggaaaggccggaagcggccgggctggatcaggatgagagatcatcttcagacaccaacgaaagtgaaataaagtcaaatgaagagccactcctaagaaagagttctcgccggtttgtcatctttccaatccagtaccctgatatttggaaaatgtataaacaggcacaggcttccttctggacagcagaagaggtcgacttatcaaaggatctccctcactggaacaagcttaaagcagatgagaagtacttcatctctcacatcttagccttttttgcagccagtgatggaattgtaaatgaaaatttggtggagcgctttagtcaggaggtgcaggttccagaggctcgctgtttctatggctttcaaattctcatcgagaatgttcactcagagatgtacagtttgctgatagacacttacatcagagatcccaagaaaagggaatttttatttaatgcaattgaaaccatgccctatgttaagaaaaaagcagattgggccttgcgatggatagcagatagaaaatctacttttggggaaagagtggtggcctttgctgctgtagaaggagttttcttctcaggatcttttgctgctatattctggctaaagaagagaggtcttatgccaggactcactttttccaatgaactcatcagcagagatgaaggacttcactgtgactttgcttgcctgatgttccaatacttagtaaataagccttcagaagaaagggtcagggagatcattgttgatgctgtcaaaattgagcaggagtttttaacagaagccttgccagttggcctcattggaatgaattgcattttgatgaaacagtacattgagtttgtagctgacagattacttgtggaacttggattctcaaaggtttttcaggcagaaaatccttttgattttatggaaaacatttctttagaaggaaaaacaaatttctttgagaaacgagtttcagagtatcagcgttttgcagttatggcagaaaccacagataacgtcttcaccttggatgcagatttttaa

SEQ ID NO: 17 - protein, 351 aa, Homo sapiens
MGDPERPEAAGLDQDERSSSDTNESEIKSNEEPLLRKSSRRFVIFPIQYPDIWKMYKQAQASFWTAEEVDLSKDLPHWNKLKADEKYFISHILAFFAASDGIVNENLVERFSQEVQVPEARCFFGFQILIENVHSEMYSLLIDTYIRDPKKREFLFNAIETMPYVKKKADWALRWIADRKSTFGERVVAFAAVEGVFFSGSFAAIFWLKKRGLMPGLTFSNELISRDEGLHCDFACLMFQYLVNKPSEERVREIIVDAVKIEQEFLTEALPVGLIGMNCILMKQYIEFVADRLLVELGFSKVFQAENPFDFMENISLEGKTNFFEKRVSEYQRFAVMAETTDNVFTLDADF

SEQ ID NO: 18 - DNA, 1056 bp, Homo sapiens
atgggcgacccggaaaggccggaagcggccgggctggatcaggatgagagatcatcttcagacaccaacgaaagtgaaataaagtcaaatgaagagccactcctaagaaagagttctcgccggtttgtcatctttccaatccagtaccctgatatttggaaaatgtataaacaggcacaggcttccttctggacagcagaagaggtcgacttatcaaaggatctccctcactggaacaagcttaaagcagatgagaagtacttcatctctcacatcttagccttttttgcagccagtgatggaattgtaaatgaaaatttggtggagcgctttagtcaggaggtgcaggttccagaggctcgctgtttctttggctttcaaattctcatcgagaatgttcactcagagatgtacagtttgctgatagacacttacatcagagatcccaagaaaagggaatttttatttaatgcaattgaaaccatgccctatgttaagaaaaaagcagattgggccttgcgatggatagcagatagaaaatctacttttggggaaagagtggtggcctttgctgctgtagaaggagttttcttctcaggatcttttgctgctatattctggctaaagaagagaggtcttatgccaggactcactttttccaatgaactcatcagcagagatgaaggacttcactgtgactttgcttgcctgatgttccaatacttagtaaataagccttcagaagaaagggtcagggagatcattgttgatgctgtcaaaattgagcaggagtttttaacagaagccttgccagttggcctcattggaatgaattgcattttgatgaaacagtacattgagtttgtagctgacagattacttgtggaacttggattctcaaaggtttttcaggcagaaaatccttttgattttatggaaaacatttctttagaaggaaaaacaaatttctttgagaaacgagtttcagagtatcagcgttttgcagttatggcagaaaccacagataacgtcttcaccttggatgcagatttttaa

SEQ ID NO: 19 - protein, 351 aa, Homo sapiens
MGDPERPEAAGLDQDERSSSDTNESEIKSNEEPLLRKSSRRFVIFPIQYPDIWKMYKQAQASFWTAEEVDLSKDLPHWNKLKADEKYFISHILAFFAASDGIVNENLVERFSQEVQVPEARCFWGFQILIENVHSEMYSLLIDTYIRDPKKREFLFNAIETMPYVKKKADWALRWIADRKSTFGERVVAFAAVEGVFFSGSFAAIFWLKKRGLMPGLTFSNELISRDEGLHCDFACLMFQYLVNKPSEERVREIIVDAVKIEQEFLTEALPVGLIGMNCILMKQYIEFVADRLLVELGFSKVFQAENPFDFMENISLEGKTNFFEKRVSEYQRFAVMAETTDNVFTLDADF

SEQ ID NO: 20 - DNA, 1056 bp, Homo sapiens
atgggcgacccggaaaggccggaagcggccgggctggatcaggatgagagatcatcttcagacaccaacgaaagtgaaataaagtcaaatgaagagccactcctaagaaagagttctcgccggtttgtcatctttccaatccagtaccctgatatttggaaaatgtataaacaggcacaggcttccttctggacagcagaagaggtcgacttatcaaaggatctccctcactggaacaagcttaaagcagatgagaagtacttcatctctcacatcttagccttttttgcagccagtgatggaattgtaaatgaaaatttggtggagcgctttagtcaggaggtgcaggttccagaggctcgctgtttctggggctttcaaattctcatcgagaatgttcactcagagatgtacagtttgctgatagacacttacatcagagatcccaagaaaagggaatttttatttaatgcaattgaaaccatgccctatgttaagaaaaaagcagattgggccttgcgatggatagcagatagaaaatctacttttggggaaagagtggtggcctttgctgctgtagaaggagttttcttctcaggatcttttgctgctatattctggctaaagaagagaggtcttatgccaggactcactttttccaatgaactcatcagcagagatgaaggacttcactgtgactttgcttgcctgatgttccaatacttagtaaataagccttcagaagaaagggtcagggagatcattgttgatgctgtcaaaattgagcaggagtttttaacagaagccttgccagttggcctcattggaatgaattgcattttgatgaaacagtacattgagtttgtagctgacagattacttgtggaacttggattctcaaaggtttttcaggcagaaaatccttttgattttatggaaaacatttctttagaaggaaaaacaaatttctttgagaaacgagtttcagagtatcagcgttttgcagttatggcagaaaccacagataacgtcttcaccttggatgcagatttttaa

SEQ ID NO: 21 - protein, 351 aa, Homo sapiens
MGDPERPEAAGLDQDERSSSDTNESEIKSNEEPLLRKSSRRFVIFPIQYPDIWKMYKQAQASFWTAEEVDLSKDLPHWNKLKADEKYFISHILAFFAASDGIVNENLVERFSQEVQVPEARCFFGFQILIENVHSEMFSLLIDTYIRDPKKREFLFNAIETMPYVKKKADWALRWIADRKSTFGERVVAFAAVEGVFFSGSFAAIFWLKKRGLMPGLTFSNELISRDEGLHCDFACLMFQYLVNKPSEERVREIIVDAVKIEQEFLTEALPVGLIGMNCILMKQYIEFVADRLLVELGFSKVFQAENPFDFMENISLEGKTNFFEKRVSEYQRFAVMAETTDNVFTLDADF

SEQ ID NO: 22 - DNA, 1056 bp, Homo sapiens
atgggcgacccggaaaggccggaagcggccgggctggatcaggatgagagatcatcttcagacaccaacgaaagtgaaataaagtcaaatgaagagccactcctaagaaagagttctcgccggtttgtcatctttccaatccagtaccctgatatttggaaaatgtataaacaggcacaggcttccttctggacagcagaagaggtcgacttatcaaaggatctccctcactggaacaagcttaaagcagatgagaagtacttcatctctcacatcttagccttttttgcagccagtgatggaattgtaaatgaaaatttggtggagcgctttagtcaggaggtgcaggttccagaggctcgctgtttctttggctttcaaattctcatcgagaatgttcactcagagatgtttagtttgctgatagacacttacatcagagatcccaagaaaagggaatttttatttaatgcaattgaaaccatgccctatgttaagaaaaaagcagattgggccttgcgatggatagcagatagaaaatctacttttggggaaagagtggtggcctttgctgctgtagaaggagttttcttctcaggatcttttgctgctatattctggctaaagaagagaggtcttatgccaggactcactttttccaatgaactcatcagcagagatgaaggacttcactgtgactttgcttgcctgatgttccaatacttagtaaataagccttcagaagaaagggtcagggagatcattgttgatgctgtcaaaattgagcaggagtttttaacagaagccttgccagttggcctcattggaatgaattgcattttgatgaaacagtacattgagtttgtagctgacagattacttgtggaacttggattctcaaaggtttttcaggcagaaaatccttttgattttatggaaaacatttctttagaaggaaaaacaaatttctttgagaaacgagtttcagagtatcagcgttttgcagttatggcagaaaccacagataacgtcttcaccttggatgcagatttttaa

SEQ ID NO: 23 - protein, 351 aa, Homo sapiens
MGDPERPEAAGLDQDERSSSDTNESEIKSNEEPLLRKSSRRFVIFPIQYPDIWKMYKQAQASFWTAEEVDLSKDLPHWNKLKADEKYFISHILAFFAASDGIVNENLVERFSQEVQVPEARCFFGFQILIENVHSEMWSLLIDTYIRDPKKREFLFNAIETMPYVKKKADWALRWIADRKSTFGERVVAFAAVEGVFFSGSFAAIFWLKKRGLMPGLTFSNELISRDEGLHCDFACLMFQYLVNKPSEERVREIIVDAVKIEQEFLTEALPVGLIGMNCILMKQYIEFVADRLLVELGFSKVFQAENPFDFMENISLEGKTNFFEKRVSEYQRFAVMAETTDNVFTLDADF

SEQ ID NO: 24 - DNA, 1056 bp, Homo sapiens
atgggcgacccggaaaggccggaagcggccgggctggatcaggatgagagatcatcttcagacaccaacgaaagtgaaataaagtcaaatgaagagccactcctaagaaagagttctcgccggtttgtcatctttccaatccagtaccctgatatttggaaaatgtataaacaggcacaggcttccttctggacagcagaagaggtcgacttatcaaaggatctccctcactggaacaagcttaaagcagatgagaagtacttcatctctcacatcttagccttttttgcagccagtgatggaattgtaaatgaaaatttggtggagcgctttagtcaggaggtgcaggttccagaggctcgctgtttctttggctttcaaattctcatcgagaatgttcactcagagatgtggagtttgctgatagacacttacatcagagatcccaagaaaagggaatttttatttaatgcaattgaaaccatgccctatgttaagaaaaaagcagattgggccttgcgatggatagcagatagaaaatctacttttggggaaagagtggtggcctttgctgctgtagaaggagttttcttctcaggatcttttgctgctatattctggctaaagaagagaggtcttatgccaggactcactttttccaatgaactcatcagcagagatgaaggacttcactgtgactttgcttgcctgatgttccaatacttagtaaataagccttcagaagaaagggtcagggagatcattgttgatgctgtcaaaattgagcaggagtttttaacagaagccttgccagttggcctcattggaatgaattgcattttgatgaaacagtacattgagtttgtagctgacagattacttgtggaacttggattctcaaaggtttttcaggcagaaaatccttttgattttatggaaaacatttctttagaaggaaaaacaaatttctttgagaaacgagtttcagagtatcagcgttttgcagttatggcagaaaccacagataacgtcttcaccttggatgcagatttttaa

SEQ ID NO: 25 - protein, 351 aa, Homo sapiens
MGDPERPEAAGLDQDERSSSDTNESEIKSNEEPLLRKSSRRFVIFPIQYPDIWKMYKQAQASFWTAEEVDLSKDLPHWNKLKADEKYFISHILAFFAASDGIVNENLVERFSQEVQVPEARCFWGFQILIENVHSEMFSLLIDTYIRDPKKREFLFNAIETMPYVKKKADWALRWIADRKSTFGERVVAFAAVEGVFFSGSFAAIFWLKKRGLMPGLTFSNELISRDEGLHCDFACLMFQYLVNKPSEERVREIIVDAVKIEQEFLTEALPVGLIGMNCILMKQYIEFVADRLLVELGFSKVFQAENPFDFMENISLEGKTNFFEKRVSEYQRFAVMAETTDNVFTLDADF

SEQ ID NO: 26 - DNA, 1056 bp, Homo sapiens
atgggcgacccggaaaggccggaagcggccgggctggatcaggatgagagatcatcttcagacaccaacgaaagtgaaataaagtcaaatgaagagccactcctaagaaagagttctcgccggtttgtcatctttccaatccagtaccctgatatttggaaaatgtataaacaggcacaggcttccttctggacagcagaagaggtcgacttatcaaaggatctccctcactggaacaagcttaaagcagatgagaagtacttcatctctcacatcttagccttttttgcagccagtgatggaattgtaaatgaaaatttggtggagcgctttagtcaggaggtgcaggttccagaggctcgctgtttctggggctttcaaattctcatcgagaatgttcactcagagatgtttagtttgctgatagacacttacatcagagatcccaagaaaagggaatttttatttaatgcaattgaaaccatgccctatgttaagaaaaaagcagattgggccttgcgatggatagcagatagaaaatctacttttggggaaagagtggtggcctttgctgctgtagaaggagttttcttctcaggatcttttgctgctatattctggctaaagaagagaggtcttatgccaggactcactttttccaatgaactcatcagcagagatgaaggacttcactgtgactttgcttgcctgatgttccaatacttagtaaataagccttcagaagaaagggtcagggagatcattgttgatgctgtcaaaattgagcaggagtttttaacagaagccttgccagttggcctcattggaatgaattgcattttgatgaaacagtacattgagtttgtagctgacagattacttgtggaacttggattctcaaaggtttttcaggcagaaaatccttttgattttatggaaaacatttctttagaaggaaaaacaaatttctttgagaaacgagtttcagagtatcagcgttttgcagttatggcagaaaccacagataacgtcttcaccttggatgcagatttttaa

SEQ ID NO: 27 - protein, 351 aa, Homo sapiens
MGDPERPEAAGLDQDERSSSDTNESEIKSNEEPLLRKSSRRFVIFPIQYPDIWKMYKQAQASFWTAEEVDLSKDLPHWNKLKADEKYFISHILAFFAASDGIVNENLVERFSQEVQVPEARCFWGFQILIENVHSEMWSLLIDTYIRDPKKREFLFNAIETMPYVKKKADWALRWIADRKSTFGERVVAFAAVEGVFFSGSFAAIFWLKKRGLMPGLTFSNELISRDEGLHCDFACLMFQYLVNKPSEERVREIIVDAVKIEQEFLTEALPVGLIGMNCILMKQYIEFVADRLLVELGFSKVFQAENPFDFMENISLEGKTNFFEKRVSEYQRFAVMAETTDNVFTLDADF

SEQ ID NO: 28 - DNA, 1056 bp, Homo sapiens
atgggcgacccggaaaggccggaagcggccgggctggatcaggatgagagatcatcttcagacaccaacgaaagtgaaataaagtcaaatgaagagccactcctaagaaagagttctcgccggtttgtcatctttccaatccagtaccctgatatttggaaaatgtataaacaggcacaggcttccttctggacagcagaagaggtcgacttatcaaaggatctccctcactggaacaagcttaaagcagatgagaagtacttcatctctcacatcttagccttttttgcagccagtgatggaattgtaaatgaaaatttggtggagcgctttagtcaggaggtgcaggttccagaggctcgctgtttctggggctttcaaattctcatcgagaatgttcactcagagatgtggagtttgctgatagacacttacatcagagatcccaagaaaagggaatttttatttaatgcaattgaaaccatgccctatgttaagaaaaaagcagattgggccttgcgatggatagcagatagaaaatctacttttggggaaagagtggtggcctttgctgctgtagaaggagttttcttctcaggatcttttgctgctatattctggctaaagaagagaggtcttatgccaggactcactttttccaatgaactcatcagcagagatgaaggacttcactgtgactttgcttgcctgatgttccaatacttagtaaataagccttcagaagaaagggtcagggagatcattgttgatgctgtcaaaattgagcaggagtttttaacagaagccttgccagttggcctcattggaatgaattgcattttgatgaaacagtacattgagtttgtagctgacagattacttgtggaacttggattctcaaaggtttttcaggcagaaaatccttttgattttatggaaaacatttctttagaaggaaaaacaaatttctttgagaaacgagtttcagagtatcagcgttttgcagttatggcagaaaccacagataacgtcttcaccttggatgcagatttttaa

SEQ ID NO: 29 - protein, 792 aa, Homo sapiens
MHVIKRDGRQERVMFDKITSRIQKLCYGLNMDFVDPAQITMKVIQGLYSGVTTVELDTLAAETAATLTTKHPDYAILAARIAVSNLHKETKKVFSDVMEDLYNYINPHNGKHSPMVAKSTLDIVLANKDRLNSAIIYDRDFSYNYFGFKTLERSYLLKINGKVAERPQHMLMRVSVGIHKEDIDAAIETYNLLSERWFTHASPTLFNAGTNRPQLSSCFLLSMKDDSIEGIYDTLKQCALISKSAGGIGVAVSCIRATGSYIAGTNGNSNGLVPMLRVYNNTARYVDQGGNKRPGAFAIYLEPWHLDIFEFLDLKKNTGKEEQRARDLFFALWIPDLFMKRVETNQDWSLMCPNECPGLDEVWGEEFEKLYASYEKQGRVRKVVKAQQLWYAIIESQTETGTPYMLYKDSCNRKSNQQNLGTIKCSNLCTEIVEYTSKDEVAVCNLASLALNMYVTSEHTYDFKKLAEVTKVVVRNLNKIIDINYYPVPEACLSNKRHRPIGIGVQGLADAFILMRYPFESAEAQLLNKQIFETIYYGALEASCDLAKEQGPYETYEGSPVSKGILQYDMWNVTPTDLWDWKVLKEKIAKYGIRNSLLIAPMPTASTAQILGNNESIEPYTSNIYTRRVLSGEFQIVNPHLLKDLTERGLWHEEMKNQIIACNGSIQSIPEIPDDLKQLYKTVWEISQKTVLKMAAERGAFIDQSQSLNIHIAEPNYGKLTSMHFYGWKQGLKTGMYYLRTRPAANPIQFTLNKEKLKDKEKVSKEEEEKERNTAAMVCSLENRDECLMCGS

SEQ ID NO: 30 - DNA, 2379 bp, Homo sapiens
atgcatgtgatcaagcgagatggccgccaagaacgagtcatgtttgacaaaattacatctcgaatccagaagctttgttatggactcaatatggattttgttgatcctgctcagatcaccatgaaagtaatccaaggcttgtacagtggggtcaccacagtggaactagatactttggctgctgaaacagctgcaaccttgactactaagcaccctgactatgctatcctggcagccaggatcgctgtctctaacttgcacaaagaaacaaagaaagtgttcagtgatgtgatggaagacctctataactacataaatccacataatggcaaacactctcccatggtggccaagtcaacattggatattgttctggccaataaagatcgcctgaattctgctattatctatgaccgagatttctcttacaattacttcggctttaagacgctagagcggtcttatttgttgaagatcaatggaaaagtggctgaaagaccacaacatatgttgatgagagtatctgttgggatccacaaagaagacattgatgcagcaattgaaacatataatcttctttctgagaggtggtttactcatgcttcgcccactctcttcaatgctggtaccaaccgcccacaactttctagctgttttcttctgagtatgaaagatgacagcattgaaggcatttatgacactctaaagcaatgtgcattgatttctaagtctgctggaggaattggtgttgctgtgagttgtattcgggctactggcagctacattgctgggactaatggcaattccaatggccttgtaccgatgctgagagtatataacaacacagctagatatgtggatcaaggtgggaacaagcgtcctggggcatttgctatttacctggagccttggcatttagacatctttgaattccttgatttaaagaagaacacaggaaaggaagagcagcgtgccagagatcttttctttgctctttggattccggatctcttcatgaaacgagtggagactaatcaggactggtctttgatgtgtccaaatgagtgtcctggtctggatgaggtttggggagaggaatttgagaaactatatgcaagttatgagaaacaaggtcgtgtccgcaaagttgtaaaagctcagcagctttggtatgccatcattgagtctcagacggaaacaggcaccccgtatatgctctacaaagattcctgtaatcgaaagagcaaccagcagaacctgggaaccatcaaatgcagcaacctgtgcacagaaatagtggagtacaccagcaaagatgaggttgctgtttgtaatttggcttccctggccctgaatatgtatgtcacatcagaacacacatacgactttaagaagttggctgaagtcactaaagtcgttgtccgaaacttgaataaaattattgatataaactactatcctgtaccagaggcatgcctatcaaataaacgccatcgccccattggaattggggtacaaggtctggcagatgcttttatcctgatgagatacccttttgagagtgcagaagcccagttactgaataagcagatctttgaaactatttattatggtgctctggaagccagctgtgaccttgccaaggagcagggcccatacgaaacctatgagggctctccagttagcaaaggaattcttcagtatgatatgtggaatgttactcctacagacctatgggactggaaggttctcaaggagaagattgcaaagtatggtataagaaacagtttacttattgccccgatgcctacagcttccactgctcagatcctggggaataatgagtccattgaaccttacaccagcaacatctatactcgcagagtcttgtcaggagaatttcagattgtaaatcctcacttattgaaagatcttaccgagcggggcctatggcatgaagagatgaaaaaccagattattgcatgcaatggctctattcagagcataccagaaattcctgatgacctgaagcaactttataaaactgtgtgggaaatctctcagaaaactgttctcaagatggcagctgagagaggtgctttcattgatcaaagccaatctttgaacatccacattgctgagcctaactatggcaaactcactagtatgcacttctacggctggaagcagggtttgaagactgggatgtattatttaaggacaagaccagcagctaatccaatccagttcactctaaataaggagaagctaaaagataaagaaaaggtatcaaaagaggaagaagagaaggagaggaacacagcagccatggtgtgctctttggagaatagagatgaatgtctgatgtgtggatcctga

SEQ ID NO: 31 - protein, 15 aa, Homo sapiens
YGFQIAMENIHSEMY

SEQ ID NO: 32 - DNA, 45 bp, Homo sapiens
tatggcttccaaattgccatggaaaacatacattctgaaatgtat

SEQ ID NO: 33 - protein, 15 aa, Homo sapiens
YGFQILIENVHSEMY

SEQ ID NO: 34 - DNA, 33 bp, Homo sapiens
tatggctttcaaattctcatcgagaatgttcac

SEQ ID NO: 35 - protein, 132 aa, Homo sapiens
DGIVNENLVERFSQEVQITEARCFYGFQIAMENIHSEMYSLLIDTYIKDPKEREFLFNAIETMPCVKKKADWALRWIGDKEATYGERVVAFAAVEGIFFSGSFASIFWLKKRGLMPGLTFSNELISRDEGLH

SEQ ID NO: 36 - DNA, 396 bp, Homo sapiens
gatggcatagtaaatgaaaacttggtggagcgatttagccaagaagttcagattacagaagcccgctgtttctatggcttccaaattgccatggaaaacatacattctgaaatgtatagtcttcttattgacacttacataaaagatcccaaagaaagggaatttctcttcaatgccattgaaacgatgccttgtgtcaagaagaaggcagactgggccttgcgctggattggggacaaagaggctacctatggtgaacgtgttgtagcctttgctgcagtggaaggcattttcttttccggttcttttgcgtcgatattctggctcaagaaacgaggactgatgcctggcctcacattttctaatgaacttattagcagagatgagggtttacac

SEQ ID NO: 37 - protein, 132 aa, Homo sapiens
DGIVNENLVERFSQEVQVPEARCFYGFQILIENVHSEMYSLLIDTYIRDPKKREFLFNAIETMPYVKKKADWALRWIADRKSTFGERVVAFAAVEGVFFSGSFAAIFWLKKRGLMPGLTFSNELISRDEGLH

SEQ ID NO: 38 - DNA, 396 bp, Homo sapiens
gatggaattgtaaatgaaaatttggtggagcgctttagtcaggaggtgcaggttccagaggctcgctgtttctatggctttcaaattctcatcgagaatgttcactcagagatgtacagtttgctgatagacacttacatcagagatcccaagaaaagggaatttttatttaatgcaattgaaaccatgccctatgttaagaaaaaagcagattgggccttgcgatggatagcagatagaaaatctacttttggggaaagagtggtggcctttgctgctgtagaaggagttttcttctcaggatcttttgctgctatattctggctaaagaagagaggtcttatgccaggactcactttttccaatgaactcatcagcagagatgaaggacttcac

SEQ ID NO: 39 - protein, 208 aa, Homo sapiens
YGFQIAMENIHSEMYSLLIDTYIKDPKEREFLFNAIETMPCVKKKADWALRWIGDKEATYGERVVAFAAVEGIFFSGSFASIFWLKKRGLMPGLTFSNELISRDEGLHCDFACLMFKHLVHKPSEERVREIIINAVRIEQEFLTEALPVKLIGMNCTLMKQYIEFVADRLMLELGFSKVFRVENPFDFMENISLEGKTNFFEKRVGEY

SEQ ID NO: 40 - DNA, 624 bp, Homo sapiens
tatggcttccaaattgccatggaaaacatacattctgaaatgtatagtcttcttattgacacttacataaaagatcccaaagaaagggaatttctcttcaatgccattgaaacgatgccttgtgtcaagaagaaggcagactgggccttgcgctggattggggacaaagaggctacctatggtgaacgtgttgtagcctttgctgcagtggaaggcattttcttttccggttcttttgcgtcgatattctggctcaagaaacgaggactgatgcctggcctcacattttctaatgaacttattagcagagatgagggtttacactgtgattttgcttgcctgatgttcaaacacctggtacacaaaccatcggaggagagagtaagagaaataattatcaatgctgttcggatagaacaggagttcctcactgaggccttgcctgtgaagctcattgggatgaattgcactctaatgaagcaatacattgagtttgtggcagacagacttatgctggaactgggttttagcaaggttttcagagtagagaacccatttgactttatggagaatatttcactggaaggaaagactaacttctttgagaagagagtaggcgagtat

SEQ ID NO: 41 - protein, 208 aa, Homo sapiens
YGFQILIENVHSEMYSLLIDTYIRDPKKREFLFNAIETMPYVKKKADWALRWIADRKSTFGERVVAFAAVEGVFFSGSFAAIFWLKKRGLMPGLTFSNELISRDEGLHCDFACLMFQYLVNKPSEERVREIIVDAVKIEQEFLTEALPVGLIGMNCILMKQYIEFVADRLLVELGFSKVFQAENPFDFMENISLEGKTNFFEKRVSEY

SEQ ID NO: 42 - DNA, 624 bp, Homo sapiens
tatggctttcaaattctcatcgagaatgttcactcagagatgtacagtttgctgatagacacttacatcagagatcccaagaaaagggaatttttatttaatgcaattgaaaccatgccctatgttaagaaaaaagcagattgggccttgcgatggatagcagatagaaaatctacttttggggaaagagtggtggcctttgctgctgtagaaggagttttcttctcaggatcttttgctgctatattctggctaaagaagagaggtcttatgccaggactcactttttccaatgaactcatcagcagagatgaaggacttcactgtgactttgcttgcctgatgttccaatacttagtaaataagccttcagaagaaagggtcagggagatcattgttgatgctgtcaaaattgagcaggagtttttaacagaagccttgccagttggcctcattggaatgaattgcattttgatgaaacagtacattgagtttgtagctgacagattacttgtggaacttggattctcaaaggtttttcaggcagaaaatccttttgattttatggaaaacatttctttagaaggaaaaacaaatttctttgagaaacgagtttcagagtat

SEQ ID NO: 43 - DNA, 2500 bp, Homo sapiens
cccaggcgcagccaatgggaagggtcggaggcatggcacagccaatgggaagggccggggcaccaaagccaatgggaagggccgggagcgcgcggcgcgggagatttaaaggctgctggagtgaggggtcgcccgtgcaccctgtcccagccgtcctgtcctggctgctcgctctgcttcgctgcgcctccactatgctctccctccgtgtcccgctcgcgcccatcacggacccgcagcagctgcagctctcgccgctgaaggggctcagcttggtcgacaaggagaacacgccgccggccctgagcgggacccgcgtcctggccagcaagaccgcgaggaggatcttccaggagcccacggagccgaaaactaaagcagctgcccccggcgtggaggatgagccgctgctgagagaaaacccccgccgctttgtcatcttccccatcgagtaccatgatatctggcagatgtataagaaggcagaggcttccttttggaccgccgaggaggttgacctctccaaggacattcagcactgggaatccctgaaacccgaggagagatattttatatcccatgttctggctttctttgcagcaagcgatggcatagtaaatgaaaacttggtggagcgatttagccaagaagttcagattacagaagcccgctgtttctatggcttccaaattgccatggaaaacatacattctgaaatgtatagtcttcttattgacacttacataaaagatcccaaagaaagggaatttctcttcaatgccattgaaacgatgccttgtgtcaagaagaaggcagactgggccttgcgctggattggggacaaagaggctacctatggtgaacgtgttgtagcctttgctgcagtggaaggcattttcttttccggttcttttgcgtcgatattctggctcaagaaacgaggactgatgcctggcctcacattttctaatgaacttattagcagagatgagggtttacactgtgattttgcttgcctgatgttcaaacacctggtacacaaaccatcggaggagagagtaagagaaataattatcaatgctgttcggatagaacaggagttcctcactgaggccttgcctgtgaagctcattgggatgaattgcactctaatgaagcaatacattgagtttgtggcagacagacttatgctggaactgggttttagcaaggttttcagagtagagaacccatttgactttatggagaatatttcactggaaggaaagactaacttctttgagaagagagtaggcgagtatcagaggatgggagtgatgtcaagtccaacagagaattcttttaccttggatgctgacttctaaatgaactgaagatgtgcccttacttggctgattttttttttccatctcataagaaaaatcagctgaagtgttaccaactagccacaccatgaattgtccgtaatgttcattaacagcatctttaaaactgtgtagctacctcacaaccagtcctgtctgtttatagtgctggtagtatcaccttttgccagaaggcctggctggctgtgacttaccatagcagtgacaatggcagtcttggctttaaagtgaggggtgaccctttagtgagcttagcacagcgggattaaacagtcctttaaccagcacagccagttaaaagatgcagcctcactgcttcaacgcagattttaatgtttacttaaatataaacctggcactttacaaacaaataaacattgttttgtactcacggcggcgataatagcttgatttatttggtttctacaccaaatacattctcctgaccactaatgggagccaattcacaattcactaagtgactaaagtaagttaaacttgtgtagactaagcatgtaatttttaagttttattttaatgaattaaaatatttgttaaccaactttaaagtcagtcctgtgtatacctagatattagtcagttggtgccagatagaagacaggttgtgtttttatcctgtggcttgtgtagtgtcctgggattctctgccccctctgagtagagtgttgtgggataaaggaatctctcagggcaaggagcttcttaagttaaatcactagaaatttaggggtgatctgggccttcatatgtgtgagaagccgtttcattttatttctcactgtattttcctcaacgtctggttgatgagaaaaaattcttgaagagttttcatatgtgggagctaaggtagtattgtaaaatttcaagtcatccttaaacaaaatgatccacctaagatcttgcccctgttaagtggtgaaatcaactagaggtggttcctacaagttgttcattctagttttgtttggtgtaagtaggttgtgtgagttaattcatttatatttactatgtctgttaaatcagaaattttttattatctatgttcttctagattttacctgtagttcataaaaaaaaaaaaaaaaaaaaaaaaa

SEQ ID NO: 44 - DNA, 4955 bp, Homo sapiens
ggctggccgaagttaggcggagccccgaggcgggggaggcggggccgggccggcgcagggagagtcactcaatggacaggcgagaaagcaggaccggcgcggcggggcggggccggccgagtccctagagctgggggcggggcggacccagcggaccagcggaccacctgggtgctgtcgtagttggaggtggcctgaggagctcagttccctcagcgcccgtagcttcggcggagtctgcgcgatgggcgacccggaaaggccggaagcggccgggctggatcaggatgagagatcatcttcagacaccaacgaaagtgaaataaagtcaaatgaagagccactcctaagaaagagttctcgccggtttgtcatctttccaatccagtaccctgatatttggaaaatgtataaacaggcacaggcttccttctggacagcagaagaggtcgacttatcaaaggatctccctcactggaacaagcttaaagcagatgagaagtacttcatctctcacatcttagccttttttgcagccagtgatggaattgtaaatgaaaatttggtggagcgctttagtcaggaggtgcaggttccagaggctcgctgtttctatggctttcaaattctcatcgagaatgttcactcagagatgtacagtttgctgatagacacttacatcagagatcccaagaaaagggaatttttatttaatgcaattgaaaccatgccctatgttaagaaaaaagcagattgggccttgcgatggatagcagatagaaaatctacttttggggaaagagtggtggcctttgctgctgtagaaggagttttcttctcaggatcttttgctgctatattctggctaaagaagagaggtcttatgccaggactcactttttccaatgaactcatcagcagagatgaaggacttcactgtgactttgcttgcctgatgttccaatacttagtaaataagccttcagaagaaagggtcagggagatcattgttgatgctgtcaaaattgagcaggagtttttaacagaagccttgccagttggcctcattggaatgaattgcattttgatgaaacagtacattgagtttgtagctgacagattacttgtggaacttggattctcaaaggtttttcaggcagaaaatccttttgattttatggaaaacatttctttagaaggaaaaacaaatttctttgagaaacgagtttcagagtatcagcgttttgcagttatggcagaaaccacagataacgtcttcaccttggatgcagatttttaaaaaacctctcgttttaaaactctataaacttgtcattggtaaatagtagtctattttcctctgcttaaaaaaaattttaagtatatcctttaaaggactgggggtttgctcaaaaggaaatccaaaacctattctaaacaatttgcatttatataattttcctgtttaacaacaagagtgtgacctaaatgcttttgtcttgtcactgaaataaaagatggcattatgtggttaagagcatggggcgaggggtcagacatgagtctaaggttctgcccttactccagtgtgtgacccttggcaagtcagttaatcttggtaaacctcggtgtacttatctttaaaatgggagtaatagtaggtcctaaattcatagagtggatattaggattaggatgcaaaaataaatgcttaaccaacactactactgttagcaccactactaattatcattcattgataatattaattgcaatgatgttgtaataaaatactctcatttccttaaaataattgtgattctaggtcctaggatctagaattagatctttgtatttttaatgcttaggggaagaatataagtatctccttaaaaagaacataattctcattcacgcaagaataagttctttgaattccttagtatgtagtgaagaaaatttagttgttagttgctttgggaagcctacttatggagtggaaaccaggaggttatcatggtagttgaccttataagaaaaatgattcttcttcagaaattaaaaacataactattgccagatttagctctggaatgtttagaatcaggctagaatagcattttccaaagaatattctaagagctattagctcctctagatatttttttgggggaaaaaggggattctgtggtcagatgagtttgggaaatgctgaacacttcattcttctttagcaagtacagtcagtacatcaaagactgagcagttcagtggtacataaatttatctcgccctgcatattcccaacatacttaacacagatgttttttacctgttaacatctcacccagctagtgttcctcagaacaaagattggaaaaagctggccgagaaccatttatacatagaggaagggcttatggactgagaaagggagaacatggtagggattattgaatcatttcaaatttataccagcctgaatagtgtaccagcaattgacttaggctgtgtttctttatggttttaaaactcttgagctgttataagagatagttcttttaatgtgactatgcaacatgatagccaatggtgagggaaaaggaggtttctctagaagagtctgatgaaaggccgggaaccaaggtttttgagaagtctgcccctatttatttttagtaagtatcaagaggtagcctgagcctagttagagttagacctgtctttggatgaagaagtcttaatactgaaatactgaatttttaatacattattatttggtattctgtataccccttcaagcagttgtttcccattcccaacaaactgtactttatacaattctggatgctaaaacttagagattttctctttgcataaattttggctccattctttccataacaatctaatcaaaactgggagttctcaagtgaatgcaaaaggagcaggccataactttatttgttagatacactgtcagaaacttgagatcttttggcctatgataataccattaatttttgcattgcttcagtttgccaagtgtttttacatcatctcatttgatctcaaaacagcttgacagagcaactgttattgaaatattacagatggaaagaatgaggctcagggaagttaaatgacttggccaagatctgctcatcgtcactgtctgtacagtatttttttttagaggttgtaatgtctcagatttagtcctttaccatctatgttgatttgcttttgtctatttcctcattaattgaatatactttaaatatatatattaaagtatcaaaatatagagagacatttgaactgtattcaggtaatatgtttaaagatatttatatattgccatacaaaaacttaacatttaaaactgataatatctgtaatgacatcagaatgaaagaaaaaaaattgtacagtgtatattcctttgttttgaatccaaatctttttcataggtaatgacagatgccttaatgtgaagcttatttataatagcaataaacctaactggatttggatgaagaagtcttaatactgacatactggatttttaatgcactggtttgttatttggtattctatctctttttccaggcctccaggttgcacatttatttattatgttcaatactttggttcttagttcttaaagaatcaagaagttgtgtaatcttttaaaaatattatcttgcagataaagaaaaaaattaagagtgtgtttacaactgttttctcttttttacagtacatgtatttaaatcattgctataataaagttaagttcattaggaatataaaaacttgcagttctatgatagattgcatttattaaaaatgtttcattgtatcacatagaaatatggccaggaaggacttgagaagacagtttgatccattgcttttagacaggactgggttttgctgtccaattatatacaataatagtttttcttacaactaagctggccccagccttgtcttgatattaatacatgaaatttttataattgtctcattgtctcatttagaaacatccatatttttctgctttttctattgccattttttatttgtgcatgaattgattattgagaaaatgtagcagtttgcatatttaaaaattaatcattttgcattttacatttaaatatgctaacatcactgtcatagaattcccaaatttcatttgtagatactgaactaagggctaatgtcaggagctgatttttaatgataaagctgcagatgggctaaataaaagccaaattaatcctacaatcaggtattatgtttttaaaccaagttgagtgaattggtagtggacttgggaaatcttccccagcagaatctggatgaatggcacagaattgaaatctctttgtttcccaccatttccctttaagtgctctgctcctttgtaaaaagttaaagatttgaaagagaatctcatattcccgaggcattaggaagaaaggatttaatcccttcaatttggggcttaatcttgtttaaaaaaatgtaagtgaagatggaaggctggagagaatgattgctttttgtacagttaaataaggtcacaatattcttacatactttgttttacaactgtgttttcattttttcaaatgtctggccatttagcaaagttatttactatttactgtgtacatagaaagctttattatgtgtggtgtatctaaattttttttgctgaaatacattatggtcaatcaagccaagcctgcatgtacagaatttgtttttttttcaaataaattagttgttttcttatttttttggcttagtatgttgaaataaactatggtatcttcatcattttgtacatttcctttttgaggaaggtttctttataagtgcaagggctaccctaataaaggaatgtatatacttaaaaaaaaaaaaaaaaaaaaaaaaaa

SEQ ID NO: 45 - DNA, 3117 bp, Homo sapiens
attcgaaccccgtcgcgcccctttgtgcgtcacgggtggcgggcgcgggaaggggatttggattgttgcgcctctgctctgaagaaagtgctgtctggctccaactccagttctttcccctgagcagcgcctggaacctaacccttcccactctgtcaccttctcgatcccgccggcgctttagagccgcagtccagtcttggatccttcagagcctcagccactagctgcgatgcatgtgatcaagcgagatggccgccaagaacgagtcatgtttgacaaaattacatctcgaatccagaagctttgttatggactcaatatggattttgttgatcctgctcagatcaccatgaaagtaatccaaggcttgtacagtggggtcaccacagtggaactagatactttggctgctgaaacagctgcaaccttgactactaagcaccctgactatgctatcctggcagccaggatcgctgtctctaacttgcacaaagaaacaaagaaagtgttcagtgatgtgatggaagacctctataactacataaatccacataatggcaaacactctcccatggtggccaagtcaacattggatattgttctggccaataaagatcgcctgaattctgctattatctatgaccgagatttctcttacaattacttcggctttaagacgctagagcggtcttatttgttgaagatcaatggaaaagtggctgaaagaccacaacatatgttgatgagagtatctgttgggatccacaaagaagacattgatgcagcaattgaaacatataatcttctttctgagaggtggtttactcatgcttcgcccactctcttcaatgctggtaccaaccgcccacaactttctagctgttttcttctgagtatgaaagatgacagcattgaaggcatttatgacactctaaagcaatgtgcattgatttctaagtctgctggaggaattggtgttgctgtgagttgtattcgggctactggcagctacattgctgggactaatggcaattccaatggccttgtaccgatgctgagagtatataacaacacagctagatatgtggatcaaggtgggaacaagcgtcctggggcatttgctatttacctggagccttggcatttagacatctttgaattccttgatttaaagaagaacacaggaaaggaagagcagcgtgccagagatcttttctttgctctttggattccggatctcttcatgaaacgagtggagactaatcaggactggtctttgatgtgtccaaatgagtgtcctggtctggatgaggtttggggagaggaatttgagaaactatatgcaagttatgagaaacaaggtcgtgtccgcaaagttgtaaaagctcagcagctttggtatgccatcattgagtctcagacggaaacaggcaccccgtatatgctctacaaagattcctgtaatcgaaagagcaaccagcagaacctgggaaccatcaaatgcagcaacctgtgcacagaaatagtggagtacaccagcaaagatgaggttgctgtttgtaatttggcttccctggccctgaatatgtatgtcacatcagaacacacatacgactttaagaagttggctgaagtcactaaagtcgttgtccgaaacttgaataaaattattgatataaactactatcctgtaccagaggcatgcctatcaaataaacgccatcgccccattggaattggggtacaaggtctggcagatgcttttatcctgatgagatacccttttgagagtgcagaagcccagttactgaataagcagatctttgaaactatttattatggtgctctggaagccagctgtgaccttgccaaggagcagggcccatacgaaacctatgagggctctccagttagcaaaggaattcttcagtatgatatgtggaatgttactcctacagacctatgggactggaaggttctcaaggagaagattgcaaagtatggtataagaaacagtttacttattgccccgatgcctacagcttccactgctcagatcctggggaataatgagtccattgaaccttacaccagcaacatctatactcgcagagtcttgtcaggagaatttcagattgtaaatcctcacttattgaaagatcttaccgagcggggcctatggcatgaagagatgaaaaaccagattattgcatgcaatggctctattcagagcataccagaaattcctgatgacctgaagcaactttataaaactgtgtgggaaatctctcagaaaactgttctcaagatggcagctgagagaggtgctttcattgatcaaagccaatctttgaacatccacattgctgagcctaactatggcaaactcactagtatgcacttctacggctggaagcagggtttgaagactgggatgtattatttaaggacaagaccagcagctaatccaatccagttcactctaaataaggagaagctaaaagataaagaaaaggtatcaaaagaggaagaagagaaggagaggaacacagcagccatggtgtgctctttggagaatagagatgaatgtctgatgtgtggatcctgaggaaagacttggaagagaccagcatgtcttcagtagccaaactacttcttgagcatagataggtatagtgggtttgcttgaggtggtaaggctttgctggaccctgttgcaggcaaaaggagtaattgatttaaagtactgttaatgatgttaatgatttttttttaaactcatatattgggattttcaccaaaataatgcttttgaaaaaaagaaaaaaaaaacggatatattgagaatcaaagtagaagttttaggaatgcaaaataagtcatcttgcatacagggagtggttaagtaaggtttcatcacccatttagcatgcttttctgaagacttcagttttgttaaggagatttagttttactgctttgactggtgggtctctagaagcaaaactgagtgataactcatgagaagtactgataggacctttatctggatatggtcctataggttattctgaaataaagataaacatttctaagtgaaaaaaaaaaaaaaaa

SEQ ID NO: 46 - DNA, 45 bp, Artificial Sequence
  Synthetically generated oligonucleotide
attacagaagcccgctgtttctttggcttccaaattgccatggaa

SEQ ID NO: 47 - DNA, 45 bp, Artificial Sequence
  Synthetically generated oligonucleotide
gttccagaggctcgctgtttctttggctttcaaattctcatcgag

SEQ ID NO: 48 - protein, 38 aa, Chlamydia
IFKHVTNPEARQYLLRQAFEEAVHTHTFLYICESLGLD

SEQ ID NO: 49 - protein, 19 aa, Chlamydia
KNFFETRVIEYQHAASLTW

SEQ ID NO: 50 - protein, 40 aa, Escherichia coli
LLPLISIPELETWVETWAFSETIHSRSYTHIIRNIVNDPS

SEQ ID NO: 51 - protein, 20 aa, Escherichia coli
VAPQEVEVSSYLVGQIDSEV

SEQ ID NO: 52 - protein, 40 aa, Saccharomyces cerevisiae
FSTEVQIPEAKSFYGFQIMIENIHSETYSLLIDTYIKDPK

SEQ ID NO: 53 - protein, 20 aa, Saccharomyces cerevisiae
TNFFEKRVSDYQKAGVMSKS

SEQ ID NO: 54 - protein, 40 aa, Mus musculus
FSQEVQVTEARCFYGFQIAMENIHSEMYSLLIDTYIKDPK

SEQ ID NO: 55 - protein, 20 aa, Mus musculus
TNFFEKRVGEYQRMGVMSNS

SEQ ID NO: 56 - protein, 40 aa, Homo sapiens
FSQEVQITEARCFYGFQIAMENIHSEMYSLLIDTYIKDPK

SEQ ID NO: 57 - protein, 20 aa, Homo sapiens
TNFFEKRVGEYQRMGVMSSP

SEQ ID NO: 58 - protein, 40 aa, Homo sapiens
FSQEVQVPEARCFYGFQILIENVHSEMYSLLIDTYIRDPK

SEQ ID NO: 59 - protein, 20 aa, Homo sapiens
TNFFEKRVSEYQRFAVMAET

(19) United States
(12) Patent Application Publication
(10) Pub. No.: US 2005/0260637 A1
(43) Pub. Date: Nov. 24, 2005

(54) Drug screening

(76) Inventor: Yun Yen, Arcadia, CA (US)

Correspondence Address: FISH & RICHARDSON PC, P.O. BOX 1022, MINNEAPOLIS, MN, 55440-1022, US

(21) Appl. No.: 11/088,686

(22) Filed: Mar. 23, 2005

Related U.S. Application Data
(60) Provisional application No. 60/556,836, filed on Mar. 25, 2004.

(51) Int. Cl. C12Q 1/68; C07H 21/04; C12N 9/06; C12N 15/09

(52) U.S. Cl. 435/6; 435/69.1; 435/191; 435/320.1; 435/325; 536/23.2

(57) ABSTRACT

An isolated polypeptide containing (i) an immunogenic fragment of an RNR subunit (SEQ ID NO: 1 or 15) spanning Y162 or Y369 of SEQ ID NO: 1, or Y124 or Y331 of SEQ ID NO: 15; or (ii) a mutant fragment of SEQ ID NO: 1 or 15 in which at least one of the aforementioned Y residues is replaced by a non-tyrosine residue. Disclosed are related nucleic acids, expression vectors, host cells, reconstituted RNR enzymes, preparation methods, and compound screening methods. Also disclosed are RNAi agents for inhibiting expression of a gene encoding SEQ ID NO: 1 or 15. Within the scope of this invention are methods of treating a cell proliferation-associated disorder.

### RELATED APPLICATION

This application claims priority to U.S. Provisional Application Ser. No. 60/556,836, filed on Mar. 25, 2004, the content of which is incorporated by reference in its entirety.

### BACKGROUND

Ribonucleotide reductase (RNR) is a highly regulated enzyme involved in the DNA synthesis pathway. It consists of two types of subunits: M1 and M2. In human, one RNR consists of two large subunits (hRRM1) and two small subunits (two hRRM2 subunits or two p53R2 subunits). This enzyme is responsible for de novo conversion of ribonucleoside diphosphates to deoxyribonucleoside diphosphates, which are essential for DNA synthesis and repair (Cory and Sato, 1983, Mol. Cell. Biochem. 53, 257-266; and Thelander and Berg, 1986, Mol. Cell. Biol. 6, 3433-3442). As DNA synthesis is required for cell proliferation, inhibiting RNR activity may reduce unwanted cell proliferation, e.g., cancer. Thus, RNR represents an important target for cancer treatment (Nocentini, 1996, Crit. Rev. Oncol. Hematol. 22, 89-126). Nevertheless, the progress in identifying effective and specific RNR inhibitors has been slow due to lack of both a thorough understanding of the RNR structure and specific screening systems.

### SUMMARY

This invention is based, at least in part, on the discovery of a diferric-dityrosyl radical cofactor center in mammalian RNRs and an active RNR reconstituted in vitro from recombinant hRRM1 and hRRM2, or from recombinant hRRM1 and p53R2. Listed below are the polypeptide and nucleic acid sequences for hRRM1, hRPM2, and p53R2:

hRRM2 polypeptide sequence (GenBank Accession No. S25854; SEQ ID NO: 1)

| (SEQ ID NO: 1) | |
|---|---|
| 1 | mlslrvplap itdpqqlqls plkqlslvdk entppalsgt rvlasktarr ifqeptepkt |
| 61 | kaaapgvede pllrenprrf vifpieyhdi wqmykkaeas fwtaeevdls kdiqhweslk |
| 121 | peeryfishv laffaasdgi vnenlverfs qevqitearc fygfqiamen ihsemyslli |
| 181 | dtyikdpker eflfnaietm pcvkkkadwa lrwigdkeat ygervvafaa vegiffsgsf |
| 241 | asifwlkkrg lmpqltfsne lisrdeglhc dfaclmfkhl vhkpseervr eiiinavrie |
| 301 | qefltealpv kligmnctlm kqyiefvadr lmlelgfskv frvenpfdfm enislegktn |
| 361 | ffekrvgeyq rmgvmsspte nsftldadf |

hRRM2 nucleic acid sequence (GenBank Accession No. NM_{—}001034; SEQ ID NO: 43)

| (SEQ ID NO: 43) | |
|---|---|
| 1 | cccaggcgca gccaatggga agggtcggag gcatggcaca gccaatggga agggccgggg |
| 61 | caccaaagcc aatgggaagg gccgggagcg cgcggcgcgg gagatttaaa ggctgctgga |
| 121 | gtgaggggtc gcccgtgcac cctgtcccag ccgtcctgtc ctggctgctc gctctgcttc |
| 181 | gctgcgcctc cactatgctc tccctccgtg tcccgctcgc gcccatcacg gacccgcagc |
| 241 | agctgcagct ctcgccgctg aaggggctca gcttggtcga caaggagaac acgccgccgg |
| 301 | ccctgagcgg gacccgcgtc ctggccagca agaccgcgag gaggatcttc caggagccca |
| 361 | cggagccgaa aactaaagca gctgcccccg gcgtggagga tgagccgctg ctgagagaaa |
| 421 | acccccgccg ctttgtcatc ttccccatcg agtaccatga tatctggcag atgtataaga |
| 481 | aggcagaggc ttccttttgg accgccgagg aggttgacct ctccaaggac attcagcact |
| 541 | gggaatccct gaaacccgag gagagatatt ttatatccca tgttctggct ttctttgcag |
| 601 | caagcgatgg catagtaaat gaaaacttgg tggagcgatt tagccaagaa gttcagatta |
| 661 | cagaagcccg ctgtttctat ggcttccaaa ttgccatgga aaacatacat tctgaaatgt |
| 721 | atagtcttct tattgacact tacataaaag atcccaaaga aagggaattt ctcttcaatg |
| 781 | ccattgaaac gatgccttgt gtcaagaaga aggcagactg ggccttgcgc tggattgggg |
| 841 | acaaagaggc tacctatggt gaacgtgttg tagcctttgc tgcagtggaa ggcattttct |
| 901 | tttccggttc ttttgcgtcg atattctggc tcaagaaacg aggactgatg cctggcctca |
| 961 | cattttctaa tgaacttatt agcagagatg agggtttaca ctgtgatttt gcttgcctga |
| 1021 | tgttcaaaca cctggtacac aaaccatcgg aggagagagt aagagaaata attatcaatg |
| 1081 | ctgttcggat agaacaggag ttcctcactg aggccttgcc tgtgaagctc attgggatga |
| 1141 | attgcactct aatgaagcaa tacattgagt ttgtggcaga cagacttatg ctggaactgg |
| 1201 | gttttagcaa ggttttcaga gtagagaacc catttgactt tatggagaat atttcactgg |
| 1261 | aaggaaagac taacttcttt gagaagagag taggcgagta tcagaggatg ggagtgatgt |
| 1321 | caagtccaac agagaattct tttaccttgg atgctgactt ctaaatgaac tgaagatgtg |
| 1381 | cccttacttg gctgattttt tttttccatc tcataagaaa aatcagctga agtgttacca |
| 1441 | actagccaca ccatgaattg tccgtaatgt tcattaacag catctttaaa actgtgtagc |
| 1501 | tacctcacaa ccagtcctgt ctgtttatag tgctggtagt atcacctttt gccagaaggc |
| 1561 | ctggctggct gtgacttacc atagcagtga caatggcagt cttggcttta aagtgagggg |
| 1621 | tgacccttta gtgagcttag cacagcggga ttaaacagtc ctttaaccag cacagccagt |
| 1681 | taaaagatgc agcctcactg cttcaacgca gattttaatg tttacttaaa tataaacctg |
| 1741 | gcactttaca aacaaataaa cattgttttg tactcacggc ggcgataata gcttgattta |
| 1801 | tttggtttct acaccaaata cattctcctg accactaatg ggagccaatt cacaattcac |
| 1861 | taagtgacta aagtaagtta aacttgtgta gactaagcat gtaattttta agttttattt |
| 1921 | taatgaatta aaatatttgt taaccaactt taaagtcagt cctgtgtata cctagatatt |
| 1981 | agtcagttgg tgccagatag aagacaggtt gtgtttttat cctgtggctt gtgtagtgtc |
| 2041 | ctgggattct ctgccccctc tgagtagagt gttgtgggat aaaggaatct ctcagggcaa |
| 2101 | ggagcttctt aagttaaatc actagaaatt taggggtgat ctgggccttc atatgtgtga |
| 2161 | gaagccgttt cattttattt ctcactgtat tttcctcaac gtctggttga tgagaaaaaa |
| 2221 | ttcttgaaga gttttcatat gtgggagcta aggtagtatt gtaaaatttc aagtcatcct |
| 2281 | taaacaaaat gatccaccta agatcttgcc cctgttaagt ggtgaaatca actagaggtg |
| 2341 | gttcctacaa gttgttcatt ctagttttgt ttggtgtaag taggttgtgt gagttaattc |
| 2401 | atttatattt actatgtctg ttaaatcaga aattttttat tatctatgtt cttctagatt |
| 2461 | ttacctgtag ttcataaaaa aaaaaaaaaa aaaaaaaaaa |

p53R2 polypeptide sequence (GenBank Accession No. NP_{—}056528; SEQ ID NO: 15)

| (SEQ ID NO: 15) | |
|---|---|
| 1 | mgdperpeaa qldqdersss dtneseiksn eepllrkssr rfvifpiqyp diwkmykqaq |
| 61 | asfwtaeevd lskdlphwnk lkadekyfis hilaffaasd givnenlver fsqevqvpea |
| 121 | rcfyqfqili envhsemysl lidtyirdpk kreflfnaie tmpyvkkkad walrwiadrk |
| 181 | stfgervvaf aavegvffsg sfaaifwlkk rglmpgltfs nelisrdegl hcdfaclmfq |
| 241 | ylvnkpseer vreiivdavk ieqeflteal pvgligmnci lmkqyiefva drllvelgfs |
| 301 | kvfqaenpfd fmenislegk tnffekrvse yqrfavmaet tdnvftldad f |

P53R2 nucleic acid sequence (GenBank Accession No. NM_{—}015713; SEQ ID NO: 44)

| (SEQ ID NO: 44) | |
|---|---|
| 1 | ggctggccga agttaggcgg agccccgagg cgggggaggc ggggccgggc cggcgcaggg |
| 61 | agagtcactc aatggacagg cgagaaagca ggaccggcgc ggcggggcgg ggccggccga |
| 121 | gtccctagag ctgggggcgg ggcggaccca gcggaccagc ggaccacctg ggtgctgtcg |
| 181 | tagttggagg tggcctgagg agctcagttc cctcagcgcc cgtagcttcg gcggagtctg |
| 241 | cgcgatgggc gacccggaaa ggccggaagc ggccgggctg gatcaggatg agagatcatc |
| 301 | ttcagacacc aacgaaagtg aaataaagtc aaatgaagag ccactcctaa gaaagagttc |
| 361 | tcgccggttt gtcatctttc caatccagta ccctgatatt tggaaaatgt ataaacaggc |
| 421 | acaggcttcc ttctggacag cagaagaggt cgacttatca aaggatctcc ctcactggaa |
| 481 | caagcttaaa gcagatgaga agtacttcat ctctcacatc ttagcctttt ttgcagccag |
| 541 | tgatggaatt gtaaatgaaa atttggtgga gcgctttagt caggaggtgc aggttccaga |
| 601 | ggctcgctgt ttctatggct ttcaaattct catcgagaat gttcactcag agatgtacag |
| 661 | tttgctgata gacacttaca tcagagatcc caagaaaagg gaatttttat ttaatgcaat |
| 721 | tgaaaccatg ccctatgtta agaaaaaagc agattgggcc ttgcgatgga tagcagatag |
| 781 | aaaatctact tttggggaaa gagtggtggc ctttgctgct gtagaaggag ttttcttctc |
| 841 | aggatctttt gctgctatat tctggctaaa gaagagaggt cttatgccag gactcacttt |
| 901 | ttccaatgaa ctcatcagca gagatgaagg acttcactgt gactttgctt gcctgatgtt |
| 961 | ccaatactta gtaaataagc cttcagaaga aagggtcagg gagatcattg ttgatgctgt |
| 1021 | caaaattgag caggagtttt taacagaagc cttgccagtt ggcctcattg gaatgaattg |
| 1081 | cattttgatg aaacagtaca ttgagtttgt agctgacaga ttacttgtgg aacttggatt |
| 1141 | ctcaaaggtt tttcaggcag aaaatccttt tgattttatg gaaaacattt ctttagaagg |
| 1201 | aaaaacaaat ttctttgaga aacgagtttc agagtatcag cgttttgcag ttatggcaga |
| 1261 | aaccacagat aacgtcttca ccttggatgc agatttttaa aaaacctctc gttttaaaac |
| 1321 | tctataaact tgtcattggt aaatagtagt ctattttcct ctgcttaaaa aaaattttaa |
| 1381 | gtatatcctt taaaggactg ggggtttgct caaaaggaaa tccaaaacct attctaaaca |
| 1441 | atttgcattt atataatttt cctgtttaac aacaagagtg tgacctaaat gcttttgtct |
| 1501 | tgtcactgaa ataaaagatg gcattatgtg gttaagagca tggggcgagg ggtcagacat |
| 1561 | gagtctaagg ttctgccctt actccagtgt gtgacccttg gcaagtcagt taatcttggt |
| 1621 | aaacctcggt gtacttatct ttaaaatggg agtaatagta ggtcctaaat tcatagagtg |
| 1681 | gatattagga ttaggatgca aaaataaatg cttaaccaac actactactg ttagcaccac |
| 1741 | tactaattat cattcattga taatattaat tgcaatgatg ttgtaataaa atactctcat |
| 1801 | ttccttaaaa taattgtgat tctaggtcct aggatctaga attagatctt tgtattttta |
| 1861 | atgcttaggg gaagaatata agtatctcct taaaaagaac ataattctca ttcacgcaag |
| 1921 | aataagttct ttgaattcct tagtatgtag tgaagaaaat ttagttgtta gttgctttgg |
| 1981 | gaagcctact tatggagtgg aaaccaggag gttatcatgg tagttgacct tataagaaaa |
| 2041 | atgattcttc ttcagaaatt aaaaacataa ctattgccag atttagctct ggaatgttta |
| 2101 | gaatcaggct agaatagcat tttccaaaga atattctaag agctattagc tcctctagat |
| 2161 | atttttttgg gggaaaaagg ggattctgtg gtcagatgag tttgggaaat gctgaacact |
| 2221 | tcattcttct ttagcaagta cagtcagtac atcaaagact gagcagttca gtggtacata |
| 2281 | aatttatctc gccctgcata ttcccaacat acttaacaca gatgtttttt acctgttaac |
| 2341 | atctcaccca gctagtgttc ctcagaacaa agattggaaa aagctggccg agaaccattt |
| 2401 | atacatagag gaagggctta tggactgaga aagggagaac atggtaggga ttattgaatc |
| 2461 | atttcaaatt tataccagcc tgaatagtgt accagcaatt gacttaggct gtgtttcttt |
| 2521 | atggttttaa aactcttgag ctgttataag agatagttct tttaatgtga ctatgcaaca |
| 2581 | tgatagccaa tggtgaggga aaaggaggtt tctctagaag agtctgatga aaggccggga |
| 2641 | accaaggttt ttgagaagtc tgcccctatt tatttttagt aagtatcaag aggtagcctg |
| 2701 | agcctagtta gagttagacc tgtctttgga tgaagaagtc ttaatactga aatactgaat |
| 2761 | ttttaataca ttattatttg gtattctgta taccccttca agcagttgtt tcccattccc |
| 2821 | aacaaactgt actttataca attctggatg ctaaaactta gagattttct ctttgcataa |
| 2881 | attttggctc cattctttcc ataacaatct aatcaaaact gggagttctc aagtgaatgc |
| 2941 | aaaaggagca ggccataact ttatttgtta gatacactgt cagaaacttg agatcttttg |
| 3001 | gcctatgata ataccattaa tttttgcatt gcttcagttt gccaagtgtt tttacatcat |
| 3061 | ctcatttgat ctcaaaacag cttgacagag caactgttat tgaaatatta cagatggaaa |
| 3121 | gaatgaggct cagggaagtt aaatgacttg gccaagatct gctcatcgtc actgtctgta |
| 3181 | cagtattttt ttttagaggt tgtaatgtct cagatttagt cctttaccat ctatgttgat |
| 3241 | ttgcttttgt ctatttcctc attaattgaa tatactttaa atatatatat taaagtatca |
| 3301 | aaatatagag agacatttga actgtattca ggtaatatgt ttaaagatat ttatatattg |
| 3361 | ccatacaaaa acttaacatt taaaactgat aatatctgta atgacatcag aatgaaagaa |
| 3421 | aaaaaattgt acagtgtata ttcctttgtt ttgaatccaa atctttttca taggtaatga |
| 3481 | cagatgcctt aatgtgaagc ttatttataa tagcaataaa cctaactgga tttggatgaa |
| 3541 | gaagtcttaa tactgacata ctggattttt aatgcactgg tttgttattt ggtattctat |
| 3601 | ctctttttcc aggcctccag gttgcacatt tatttattat gttcaatact ttggttctta |
| 3661 | gttcttaaag aatcaagaag ttgtgtaatc ttttaaaaat attatcttgc agataaagaa |
| 3721 | aaaaattaag agtgtgttta caactgtttt ctctttttta cagtacatgt atttaaatca |
| 3781 | ttgctataat aaagttaagt tcattaggaa tataaaaact tgcagttcta tgatagattg |
| 3841 | catttattaa aaatgtttca ttgtatcaca tagaaatatg gccaggaagg acttgagaag |
| 3901 | acagtttgat ccattgcttt tagacaggac tgggttttgc tgtccaatta tatacaataa |
| 3961 | tagtttttct tacaactaag ctggccccag ccttgtcttg atattaatac atgaaatttt |
| 4021 | tataattgtc tcattgtctc atttagaaac atccatattt ttctgctttt tctattgcca |
| 4081 | ttttttattt gtgcatgaat tgattattga gaaaatgtag cagtttgcat atttaaaaat |
| 4141 | taatcatttt gcattttaca tttaaatatg ctaacatcac tgtcatagaa ttcccaaatt |
| 4201 | tcatttgtag atactgaact aagggctaat gtcaggagct gatttttaat gataaagctg |
| 4261 | cagatgggct aaataaaagc caaattaatc ctacaatcag gtattatgtt tttaaaccaa |
| 4321 | gttgagtgaa ttggtagtgg acttgggaaa tcttccccag cagaatctgg atgaatggca |
| 4381 | cagaattgaa atctctttgt ttcccaccat ttccctttaa gtgctctgct cctttgtaaa |
| 4441 | aagttaaaga tttgaaagag aatctcatat tcccgaggca ttaggaagaa aggatttaat |
| 4501 | cccttcaatt tggggcttaa tcttgtttaa aaaaatgtaa gtgaagatgg aaggctggag |
| 4561 | agaatgattg ctttttgtac agttaaataa ggtcacaata ttcttacata ctttgtttta |
| 4621 | caactgtgtt ttcatttttt caaatgtctg gccatttagc aaagttattt actatttact |
| 4681 | gtgtacatag aaagctttat tatgtgtggt gtatctaaat tttttttgct gaaatacatt |
| 4741 | atggtcaatc aagccaagcc tgcatgtaca gaatttgttt ttttttcaaa taaattagtt |
| 4801 | gttttcttat ttttttggct tagtatgttg aaataaacta tggtatcttc atcattttgt |
| 4861 | acatttcctt tttgaggaag gtttctttat aagtgcaagg gctaccctaa taaaggaatg |
| 4921 | tatatactta aaaaaaaaaa aaaaaaaaaa aaaaa |

hRRM1 polypeptide sequence (GenBank Accession No. S16680; SEQ ID NO: 29)

| (SEQ ID NO: 29) | |
|---|---|
| 1 | mhvikrdgrq ervmfdkits riqklcygln mdfvdpaqit mkviqglysg vttveldtla |
| 61 | aetaatlttk hpdyailaar iavsnlhket kkvfsdvmed lynyinphng khspmvakst |
| 121 | ldivlankdr lnsaiiydrd fsynyfqfkt lersyllkin gkvaerpqhm lmrvsvgihk |
| 181 | edidaaiety nllserwfth asptlfnagt nrpqlsscfl lsmkddsieg iydtlkqcal |
| 241 | isksaggigv avsciratqs yiagtngnsn glvpmlrvyn ntaryvdqgg nkrpgafaiy |
| 301 | lepwhldife fldlkkntgk eeqrardlff alwipdlfmk rvetnqdwsl mcpnecpgld |
| 361 | evwgeefekl yasyekqqrv rkvvkaqqlw yaiiesqtet gtpymlykds cnrksnqqnl |
| 421 | gtikcsnlct eiveytskde vavcnlasla lnmyvtseht ydfkklaevt kvvvrnlnki |
| 481 | idinyypvpe aclsnkrhrp iqiqvqglad afilmrypfe saeaqllnkq ifetiyygal |
| 541 | eascdlakeq gpyetyeqsp vskgilqydm wnvtptdlwd wkvlkekiak yqirnsllia |
| 601 | pmptastaqi lgnnesiepy tsniytrrvl sgefqivnph llkdltergl wheemknqii |
| 661 | acngsiqsip eipddlkqly ktvweisqkt vlkmaaerga fidqsqslni hiaepnyqkl |
| 721 | tsmhfygwkq qlktgmyylr trpaanpiqf tlnkeklkdk ekvskeeeek erntaamvcs |
| 781 | lenrdeclmc gs |

hRRM1 nucleic acid sequence (GenBank Accession No. NM_{—}001033; SEQ ID NO: 45)

| (SEQ ID NO: 24) | |
|---|---|
| 1 | attcgaaccc cgtcgcgccc ctttgtgcgt cacgggtggc gggcgcggga aggggatttg |
| 61 | gattgttgcg cctctgctct gaagaaagtg ctgtctggct ccaactccag ttctttcccc |
| 121 | tgagcagcgc ctggaaccta acccttccca ctctgtcacc ttctcgatcc cgccggcgct |
| 181 | ttagagccgc agtccagtct tggatccttc agagcctcag ccactagctg cgatgcatgt |
| 241 | gatcaagcga gatggccgcc aagaacgagt catgtttgac aaaattacat ctcgaatcca |
| 301 | gaagctttgt tatggactca atatggattt tgttgatcct gctcagatca ccatgaaagt |
| 361 | aatccaaggc ttgtacagtg gggtcaccac agtggaacta gatactttgg ctgctgaaac |
| 421 | agctgcaacc ttgactacta agcaccctga ctatgctatc ctggcagcca ggatcgctgt |
| 481 | ctctaacttg cacaaagaaa caaagaaagt gttcagtgat gtgatggaag acctctataa |
| 541 | ctacataaat ccacataatg gcaaacactc tcccatggtg gccaagtcaa cattggatat |
| 601 | tgttctggcc aataaagatc gcctgaattc tgctattatc tatgaccgag atttctctta |
| 661 | caattacttc ggctttaaga cgctagagcg gtcttatttg ttgaagatca atggaaaagt |
| 721 | ggctgaaaga ccacaacata tgttgatgag agtatctgtt gggatccaca aagaagacat |
| 781 | tgatgcagca attgaaacat ataatcttct ttctgagagg tggtttactc atgcttcgcc |
| 841 | cactctcttc aatgctggta ccaaccgccc acaactttct agctgttttc ttctgagtat |
| 901 | gaaagatgac agcattgaag gcatttatga cactctaaag caatgtgcat tgatttctaa |
| 961 | gtctgctgga ggaattggtg ttgctgtgag ttgtattcgg gctactggca gctacattgc |
| 1021 | tgggactaat ggcaattcca atggccttgt accgatgctg agagtatata acaacacagc |
| 1081 | tagatatgtg gatcaaggtg ggaacaagcg tcctggggca tttgctattt acctggagcc |
| 1141 | ttggcattta gacatctttg aattccttga tttaaagaag aacacaggaa aggaagagca |
| 1201 | gcgtgccaga gatcttttct ttgctctttg gattccggat ctcttcatga aacgagtgga |
| 1261 | gactaatcag gactggtctt tgatgtgtcc aaatgagtgt cctggtctgg atgaggtttg |
| 1321 | gggagaggaa tttgagaaac tatatgcaag ttatgagaaa caaggtcgtg tccgcaaagt |
| 1381 | tgtaaaagct cagcagcttt ggtatgccat cattgagtct cagacggaaa caggcacccc |
| 1441 | gtatatgctc tacaaagatt cctgtaatcg aaagagcaac cagcagaacc tgggaaccat |
| 1501 | caaatgcagc aacctgtgca cagaaatagt ggagtacacc agcaaagatg aggttgctgt |
| 1561 | ttgtaatttg gcttccctgg ccctgaatat gtatgtcaca tcagaacaca catacgactt |
| 1621 | taagaagttg gctgaagtca ctaaagtcgt tgtccgaaac ttgaataaaa ttattgatat |
| 1681 | aaactactat cctgtaccag aggcatgcct atcaaataaa cgccatcgcc ccattggaat |
| 1741 | tggggtacaa ggtctggcag atgcttttat cctgatgaga tacccttttg agagtgcaga |
| 1801 | agcccagtta ctgaataagc agatctttga aactatttat tatggtgctc tggaagccag |
| 1861 | ctgtgacctt gccaaggagc agggcccata cgaaacctat gagggctctc cagttagcaa |
| 1921 | aggaattctt cagtatgata tgtggaatgt tactcctaca gacctatggg actggaaggt |
| 1981 | tctcaaggag aagattgcaa agtatggtat aagaaacagt ttacttattg ccccgatgcc |
| 2041 | tacagcttcc actgctcaga tcctggggaa taatgagtcc attgaacctt acaccagcaa |
| 2101 | catctatact cgcagagtct tgtcaggaga atttcagatt gtaaatcctc acttattgaa |
| 2161 | agatcttacc gagcggggcc tatggcatga agagatgaaa aaccagatta ttgcatgcaa |
| 2221 | tggctctatt cagagcatac cagaaattcc tgatgacctg aagcaacttt ataaaactgt |
| 2281 | gtgggaaatc tctcagaaaa ctgttctcaa gatggcagct gagagaggtg ctttcattga |
| 2341 | tcaaagccaa tctttgaaca tccacattgc tgagcctaac tatggcaaac tcactagtat |
| 2401 | gcacttctac ggctggaagc agggtttgaa gactgggatg tattatttaa ggacaagacc |
| 2461 | agcagctaat ccaatccagt tcactctaaa taaggagaag ctaaaagata aagaaaaggt |
| 2521 | atcaaaagag gaagaagaga aggagaggaa cacagcagcc atggtgtgct ctttggagaa |
| 2581 | tagagatgaa tgtctgatgt gtggatcctg aggaaagact tggaagagac cagcatgtct |
| 2641 | tcagtagcca aactacttct tgagcataga taggtatagt gggtttgctt gaggtggtaa |
| 2701 | ggctttgctg gaccctgttg caggcaaaag gagtaattga tttaaagtac tgttaatgat |
| 2761 | gttaatgatt tttttttaaa ctcatatatt gggattttca ccaaaataat gcttttgaaa |
| 2821 | aaaagaaaaa aaaaacggat atattgagaa tcaaagtaga agttttagga atgcaaaata |
| 2881 | agtcatcttg catacaggga gtggttaagt aaggtttcat cacccattta gcatgctttt |
| 2941 | ctgaagactt cagttttgtt aaggagattt agttttactg ctttgactgg tgggtctcta |
| 3001 | gaagcaaaac tgagtgataa ctcatgagaa gtactgatag gacctttatc tggatatggt |
| 3061 | cctataggtt attctgaaat aaagataaac atttctaagt gaaaaaaaaa aaaaaaa |

In the above-listed nucleic acid sequences, underlined parts represent the coding sequences, i.e., nucleotides (nts) 195-1364 of GenBank Accession No. NM_{—}001034 (SEQ ID NO: 2); nts 245-1300 of GenBank Accession No. NM_{—}015713 (SEQ ID NO: 16); and nts 233-2611 of GenBank Accession No. NM_{—}001033 (SEQ ID NO: 30).

One aspect of this invention features an isolated polypeptide containing an immunogenic fragment of the hRRM2 polypeptide (SEQ ID NO: 1) or p53R2 polypeptide (SEQ ID NO: 15). The fragment spans residue Y162 or Y369 of SEQ ID NO: 1 or residue Y124 or Y331 of SEQ ID NO: 15 (both underlined). It is at least 10 amino acid residues in length, i.e., any number (i) between 9 and 389 (the length of SEQ ID NO: 1), exclusive, for fragments of SEQ ID NO: 1, or (ii) between 9 and 351 (the length of SEQ ID NO: 15), exclusive, for fragments of SEQ ID NO: 15. In other words, the polypeptide does not include the full-length SEQ ID NO: 1 or 15. One example of such a fragment contains Y162-Y176 of SEQ ID NO: 1 (i.e., YGFQIAMENIHSEMY; SEQ ID NO: 31) or Asp138-His269 of SEQ ID NO: 1 (i.e., DGIVNENLVERFSQEVQI TEARCFYGFQIAMENIHSEMYSLLIDTYIKDPKEREFLFNAIETMPCVKKKADWA LRWIGDKEATYGERVVAFAAVEGIFFSGSFASIFWLKKRGLMPGLTFSNELISRDE GLH; SEQ ID NO: 35). Another example of such a fragment contains Y124-Y138 of SEQ ID NO: 15 (i.e., YGFQILIENVHSEMY; SEQ ID NO: 33) or Asp100-His231 of SEQ ID NO: 15 (i.e., DGIVNENLVERFSQEVQVPEARCFYGFQILIENVHSEMYSL LIDTYIRDPKKREFLFNAIETMPYVKKKADWALRWIADRKSTFGERVVAFAAVE GVFFSGSFAAIFWLKKRGLMPGLTFSNELISRDEGLH; SEQ ID NO: 37.)

An isolated polypeptide refers to a polypeptide substantially free from naturally associated molecules, i.e., it is at least 75% (i.e., any number between 75% and 100%, inclusive) pure by dry weight. Purity can be measured by any appropriate standard method, e.g., by column chromatography, polyacrylamide gel electrophoresis, or HPLC. An isolated polypeptide of the invention can be purified from a natural source (for wild type polypeptides), produced by recombinant DNA techniques, or by chemical methods.

The invention also features an isolated polypeptide containing an mutant fragment of SEQ ID NO: 1 or 15. The mutant fragment is identical to a wild type fragment of SEQ ID NO: 1 or 15 spanning the Y162 or Y369 residue of SEQ ID NO: 1 or spanning the Y124 or Y331 residue of SEQ ID NO: 15, except that the residue at position 162 or 369 of SEQ ID NO: 1 or at position 124 or 331 of SEQ ID NO: 15 is a non-tyrosine residue. Examples of a non-tyrosine residue include a phenylalanine and a tryptophan. The wild type fragment is at least 10 amino acid residues in length, i.e., any number between 10 and 389 (the length of SEQ ID NO: 1), inclusive, for wild type fragments of SEQ ID NO: 1, or between 10 and 351 (the length of SEQ ID NO: 15), inclusive, for wild type fragments of SEQ ID NO: 15. Put differently, the isolated polypeptide can be a mutant form of full-length SEQ ID NO: 1 or 15. In one example, the wild type fragment contains SEQ ID NO: 31 or 33 mentioned above. In another example, the wild type fragment contains SEQ ID NO:35, SEQ ID NO: 37, Y162-Y369 of SEQ ID NO: 1 (SEQ ID NO: 39), Y124-Y331 of SEQ ID NO: 15 (i.e., SEQ ID NO: 41). In yet another example, the polypeptide contains a mutant of full-length SEQ ID NO: 1 or 15, in which Y162 of SEQ ID NO: 1 or Y124 of SEQ ID NO: 15 is mutated to phenylalanine (“F”) or tryptophan (“W”). Summarized in the table below are representative full-length mutants. Some of them contain a second mutation, i.e., F or W at the place of Y176 of SEQ ID NO: 1 (i.e., SEQ ID NOs: 7, 9, 11, 13) or Y138 of SEQ ID NO: 15 (i.e., SEQ ID NOs: 21, 23, 25, and 27). They can be encoded by the corresponding coding regions in SEQ ID NO: 1 or 15 except that a phenylalanine codon (TTT) or a tryptophan (TGG) replaces the codon for one of the just-mentioned tyrosines (TAT).

SEQ ID NOs.

| Full-length Mutants | Polypeptide | Nucleic acid |
|---|---|---|
| hRPM2 Y162F | 3 | 4 |
| hRPM2 Y162W | 5 | 6 |
| hRPM2 Y162F/Y176F | 7 | 8 |
| hRPM2 Y162F/Y176W | 9 | 10 |
| hRPM2 Y162W/Y176F | 11 | 12 |
| hRPM2 Y162W/Y176W | 13 | 14 |
| p53R2 Y124F | 17 | 18 |
| p53R2 Y124W | 19 | 20 |
| p53R2 Y124F/Y138F | 21 | 22 |
| p53R2 Y124F/Y138W | 23 | 24 |
| p53R2 Y124W/Y138F | 25 | 26 |
| p53R2 Y124W/Y138W | 27 | 28 |

Another aspect of the invention features an isolated nucleic acid containing a sequence encoding one of the above-described immunogenic fragments, or a complement thereof. Examples of such a nucleic acid include coding regions from GenBank Accession No. NM_{—}001034 or NM_{—}015713 that encode SEQ ID NOs: 31, 33, 35, 37,39, and 41 (i.e., SEQ ID NOs: 32, 34, 36, 38, 40, and 42, respectively). The invention also features an isolated nucleic acid containing a sequence encoding one of the above described mutant fragments, or a complement thereof. In these mutant nucleic acids, the tyrosine codon “TAT” corresponding to Y162 or Y369 of SEQ ID NO: 1 or to the Y124 or Y33 1 of SEQ ID NO: 15 is replaced by a non-tyrosine codon, such as a phenylalanine codon (e.g., TTT) or a tryptophan codon (e.g., TGG).

A nucleic acid refers to a DNA molecule (e.g., a cDNA or genomic DNA), an RNA molecule (e.g., an mRNA), or a DNA or RNA analog. A DNA or RNA analog can be synthesized from nucleotide analogs. The nucleic acid molecule can be single-stranded or double-stranded, but preferably is double-stranded DNA. An “isolated nucleic acid” refers to a nucleic acid the structure of which is not identical to that of any naturally occurring nucleic acid or to that of any fragment of a naturally occurring genomic nucleic acid. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. The nucleic acid described above can be used to express the polypeptide of this invention. For this purpose, one can operatively linked the nucleic acid to suitable regulatory sequences to generate an expression vector.

A vector refers to a nucleic acid molecule capable of transporting another nucleic acid to which it has been linked. The vector can be capable of autonomous replication or integrate into a host DNA. Examples of the vector include a plasmid, cosmid, or viral vector. The vector includes a nucleic acid in a form suitable for expression of the nucleic acid in a host cell. Preferably the vector includes one or more regulatory sequences operatively linked to the nucleic acid sequence to be expressed. A “regulatory sequence” includes promoters, enhancers, and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of protein or RNA desired, and the like. The expression vector can be introduced into host cells to produce a polypeptide of this invention. Also within the scope of this invention is a host cell that contains the above-described nucleic acid. Examples include E. coli cells, insect cells (e.g., using baculovirus expression vectors), yeast cells, or mammalian cells. See e.g., Goeddel, (1990) Gene Expression Technology: Methods in Enzymology 185, Academic Press, San Diego, Calif. To produce a polypeptide of this invention, one can culture a host cell in a medium under conditions permitting expression of the polypeptide encoded by a nucleic acid of this invention, and purify the polypeptide from the cultured cell or the medium of the cell. Alternatively, the nucleic acid of this invention can be transcribed and translated in vitro, e.g., using T7 promoter regulatory sequences and T7 polymerase.

A polypeptide of this invention can be used for identifying a compound that inhibits the enzymatic activity of RNR. Since RNR regulates cell growth, such a compound is a drug candidate for treating cell proliferation-associated disorder, e.g., cancer. The method includes contacting a compound with a polypeptide of this invention and determining binding, if any, between the compound and the polypeptide. A presence of the binding indicates that the compound is a candidate for inhibiting the RNR enzymatic activity. The compound can be a small organic molecule, a small inorganic molecule, an oligonucleotide, a peptide, a protein, or a carbohydrate. The term “small organic molecule” refers to small organic molecules other than oligonucleotides, peptides, or carbohydrates.

A polypeptide, as well as a nucleic acid, of this invention can be used to generate specific antibodies that bind specifically to an RNR subunit. More specifically, one can generate the antibodies by administering to a non-human animal the polypeptide or nucleic acid. Thus, within the scope of this invention is an immunogenic composition containing one of the aforementioned polypeptides or nucleic acids, and a pharmaceutically acceptable carrier. The composition can be used to generate polyclonal antibodies in humans or non-human animals. Monoclonal antibodies can be generated by standard techniques. Antibodies that specifically bind to RNR and inhibit the activity thereof are also drug candidates for treating a cell proliferation-associated disorder.

In a further aspect, this invention features a reconstituted dimeric RNR having a RNR activity. This reconstituted RNR contains a first purified polypeptide having the sequence of a first subunit of a naturally occurring RNR; and a second purified polypeptide containing the sequence of a second subunit of the naturally occurring RNR. The first subunit can be a large subunit, i.e., an R1 subunit. The second subunit can be a small subunit, i.e., an R2 or p53R2 subunit. A reconstituted dimeric human RNR can be used to identify a compound for treating a cell proliferation-associated disorder. The method includes incubating a compound with an Reconstituted dimeric RNR and determining a level of RNR activity. The compound is determined to be effective in treating the cell proliferation-associated disorder if the level of the RNR activity is lower than that determined in the same manner except that the compound is absent.

Also within the scope of this invention is an RNA, as well as a DNA vector encoding it, for inhibiting expression of a gene encoding SEQ ID NO: 1 or 15. The RNA contains a first nucleotide sequence that hybridizes under stringent conditions to a segment of the gene, and a second nucleotide sequence that is complementary to the first nucleotide sequence and hybridizes to the first nucleotide sequence to form a duplex structure. The term “stringent conditions” refers to conditions for hybridization in 6X sodium chloride/sodium citrate (SSC) at about 45° C., followed by one or more washes in 0.2×SSC, 0.1% SDS at 50-65° C. The first nucleotide sequence and the second nucleotide sequence can be on the same strand. They also can be on two different strands to form a double-stranded RNA. For efficient inhibition of the expression of a gene, the first nucleotide sequence is at least 19, e.g., 19-29, nts in length.

The details of one or more embodiments of the invention are set forth in the accompanying drawings and the description below. Other features, objects, and advantages of the invention will be apparent from the description and the claims.

### DETAILED DESCRIPTION

This invention relates to, among others, a number of residues of hRRM2 or p53R2. These residues, including two tyrosine residues, are involved in formation of stable radicals, which are required for RNR enzymatic activity.

It has been known that RNR requires protein free radicals for its enzymatic activity (Reichard et al., 1983, Science 221, 514-9; Jordan et al., 1998, Annu. Rev. Biochem. 67, 71-98; and Henriksen et al., 1994, J. Am. Chem. Soc. 116, 9773-9774). Based on their primary radicals, RNRs of various species have been grouped into three classes: Classes I, II, and III, (Reichard, 1993, Science 260, 1773-7). Class I RNR, found in eukaryotes, prokaryotes and viruses, is a protein complex that features two homodimers, R1 and R2. Each R1 large subunit contains a ligand binding catalytic site and a regulatory specificity site, whereas each R2 small subunit provides free radicals. Class II RNR, found mainly in bacteria, contains a transient 5′-deoxyadenosyl radical. Class III RNR, found in anaerobic bacteria, harbors a stable glycyl radical (Fontecave et al., 2002, Prog. Nucleic Acid Res. Mol. Biol. 72, 95-127).

E. coli RNR is commonly used as a model system for Class I enzymes. It has been found that each E. coli R2 subunit has only one stable tyrosyl radical at Tyr122 (corresponding to Tyr177 of the mouse counterpart). This radical “hole” is transferred to the R1 catalytic site via a long-range proton-coupled electron-transfer pathway. (Zhou et al., 2003, Cancer Res.63, 6583-94; Shao et al., 2004, Cancer Res. 64, 1-6; and Larsson et al., 1986, Embo. J. 5, 2037-40).

As shown in Example 2 below, hRRM2 or p53R2 has two tyrosine residues (Tyr176 and Tyr162 in hRRM2, Tyr138 and Tyr124 in p53R2) were located in the vicinity of and at opposite sides of a di-iron cluster. It was unexpected that two, instead of one, tyrosyl radicals are required for human RNR small subunits, hRRM2 and p53R2. Further shown in the same example, it was unexpected that a tyrosine outside the cluster, i.e., Y369 of SEQ ID NO: 1 or Y331 of SEQ ID NO: 15, is also essential for RNR activity. An RNR complex having one mutant subunit lacking one of the essential tyrosines loses the RNR activity substantially (e.g., by at least 50%, 70%, 80%, 90%, or 95%).

Accordingly, this invention features an isolated polypeptide containing (i) an immunogenic fragment that spans Y162 of SEQ ID NO: 1 or Y124 of SEQ ID NO: 15; or (ii) a mutant fragment or full-length of SEQ ID NO: 1 or 15 that lacks one of the just-described essential tyrosines. The mutant polypeptide can be generated by standard mutagenesis techniques and verified by electron paramagnetic resonance (EPR) spectroscopy and enzymatic assays as described in the examples below. Also within the scope of this invention are non-human mammalian counterparts of the above-described human wild type or mutant polypeptides.

A polypeptide of this invention can be obtained as a synthetic or recombinant polypeptide. To prepare a recombinant polypeptide, a nucleic acid encoding it can be linked to another nucleic acid encoding a fusion partner, e.g., Glutathione-S-Transferase (GST), 6×-His epitope tag, or M13 Gene 3 protein. The resultant fusion nucleic acid expresses in suitable host cells a fusion protein that can be isolated by methods known in the art. A variety of host-expression vector systems can be used. These include, but are not limited to, microorganisms such as bacteria transformed with recombinant bacteriophage DNA, plasmid DNA, or cosmid DNA expression vectors; yeast transformed with recombinant yeast expression vectors; and human cell lines infected with recombinant virus or plasmid expression vectors. Isolation and purification of recombinant polypeptides or its fragments can be carried out by conventional means including preparative chromatography and immunological separations involving monoclonal or polyclonal antibodies. The isolated fusion protein can be further treated, e.g., by enzymatic digestion, to remove the fusion partner and obtain the recombinant polypeptide of this invention.

A polypeptide of the invention can be used to generate antibodies in animals (for production of antibodies) or humans (for treatment of diseases). Methods of making monoclonal and polyclonal antibodies and fragments thereof are known in the art. See, e.g., Harlow et al., 1988, Antibodies: A Laboratory Manual, Cold Spring Harbor Laboratory, N.Y. The term “antibody” includes intact molecules and fragments thereof, such as Fab, F(ab′)₂, Fv, scFv (single chain antibody), and dAb (domain antibody; Ward et. al., 1989 Nature, 341, 544). These antibodies can be used for detecting an RNR subunit or in identifying a compound that binds to the polypeptide. Particularly, antibodies specifically binding to the above-described essential tyrosines or residues forming the cluster are useful as RNR inhibitors for treating a cell proliferation-associated disorder. Antibodies specific for the tyrosines can be identified by differential binding to the above-described mutant fragments or their wild type counterparts.

In general, a polypeptide of the invention can be coupled to a carrier protein, such as KLH, mixed with an adjuvant, and injected into a host animal. Antibodies produced in that animal can then be purified by peptide affinity chromatography. Commonly employed host animals include rabbits, mice, guinea pigs, and rats. Various adjuvants that can be used to increase the immunological response depend on the host species and include Freund's adjuvant (complete and incomplete), mineral gels such as aluminum hydroxide, surface active substances such as lysolecithin, pluronic polyols, polyanions, peptides, oil emulsions, keyhole limpet hemocyanin, and dinitrophenol. Useful human adjuvants include BCG (bacille Calmette-Guerin) and Corynebacterium parvum.

Polyclonal antibodies, heterogeneous populations of antibody molecules, are present in the sera of the immunized subjects. Monoclonal antibodies, homogeneous populations of antibodies to a polypeptide of this invention, can be prepared using standard hybridoma technology (see, for example, Kohler et al. (1975) Nature 256, 495; Kohler et al. (1976) Eur. J. Immunol. 6, 511; Kohler et al. (1976) Eur. J. Immunol. 6, 292; and Hammerling et al. (1981) Monoclonal Antibodies and T Cell Hybridomas, Elsevier, N.Y.). In particular, monoclonal antibodies can be obtained by any technique that provides for the production of antibody molecules by continuous cell lines in culture such as described in Kohler et al. (1975) Nature 256, 495 and U.S. Pat. No. 4,376,110; the human B-cell hybridoma technique (Kosbor et al. (1983) Immunol Today 4, 72; Cole et al. (1983) Proc. Natl. Acad. Sci. USA 80, 2026, and the EBV-hybridoma technique (Cole et al. (1983) Monoclonal Antibodies and Cancer Therapy, Alan R. Liss, Inc., pp. 77-96). Such antibodies can be of any immunoglobulin class including IgG, IgM, IgE, IgA, IgD, and any subclass thereof. The hybridoma producing the monoclonal antibodies of the invention may be cultivated in vitro or in vivo. The ability to produce high titers of monoclonal antibodies in vivo makes it a particularly useful method of production.

In addition, techniques developed for the production of “chimeric antibodies” can be used. See, e.g., Morrison et al. (1984) Proc. Natl. Acad. Sci. USA 81, 6851; Neuberger et al. (1984) Nature 312, 604; and Takeda et al. (1984) Nature 314:452. A chimeric antibody is a molecule in which different portions are derived from different animal species, such as those having a variable region derived from a murine monoclonal antibody and a human immunoglobulin constant region. Alternatively, techniques described for the production of single chain antibodies (U.S. Pat. Nos. 4,946,778 and 4,704,692) can be adapted to produce a phage library of single chain Fv antibodies. Single chain antibodies are formed by linking the heavy and light chain fragments of the Fv region via an amino acid bridge. Moreover, antibody fragments can be generated by known techniques. For example, such fragments include, but are not limited to, F(ab′)₂ fragments that can be produced by pepsin digestion of an antibody molecule, and Fab fragments that can be generated by reducing the disulfide bridges of F(ab′)₂ fragments. Antibodies can also be humanized by methods known in the art. For example, monoclonal antibodies with a desired binding specificity can be commercially humanized (Scotgene, Scotland; and Oxford Molecular, Palo Alto, Calif.). Fully human antibodies, such as those expressed in transgenic animals are also features of the invention (see, e.g., Green et al. (1994) Nature Genetics 7, 13; and U.S. Pat. Nos. 5,545,806 and 5,569,825).

The invention features a recombinant RNR reconstituted by purified RNR subunits. As shown in Example 1 below, it was unexpected that the reconstituted RNR maintained the RNR activity. This recombinant RNR is reconstituted by mixing large and small RNR subunits at a suitable ratio (e.g., 1:1 to 1:3). The RNR activity can then be evaluated by standard techniques. Full-length RNR large and small subunits, as well as their functional equivalents, can be used in reconstituting an active recombinant RNR. A “functional equivalent” refers to a polypeptide derivative of the RNR large or small subunit, e.g., a protein having one or more point mutations, insertions, deletions, truncations, a fusion protein, or a combination thereof Once forming a complex with other subunits, the resulting complex exhibits the activity of the wild type RNR.

The above-described polypeptide and reconstituted RNR can be used for identifying compounds that inhibit the activity of RNR in a cell-free system. Compounds thus identified can be used, e.g., for preventing and treating a cell proliferation-associated disorder. Candidate compounds (e.g., proteins, peptides, peptidomimetics, peptoids, antibodies, small molecules, or other drugs) can be obtained using any of the numerous approaches in combinatorial library methods known in the art. Such libraries include: peptide libraries, peptoid libraries (libraries of molecules having the functionalities of peptides, but with a novel, non-peptide backbone that is resistant to enzymatic degradation); spatially addressable parallel solid phase or solution phase libraries; synthetic libraries obtained by deconvolution or affinity chromatography selection; and the “one-bead one-compound” libraries. See, e.g., Zuckermann et al. 1994, J. Med. Chem. 37:2678-2685; and Lam, 1997, Anticancer Drug Des. 12:145. Examples of methods for the synthesis of molecular libraries can be found in, e.g., DeWitt et al., 1993, PNAS USA 90:6909; Erb et al., 1994, PNAS USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994 J. Med. Chem. 37:1233. Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria (U.S. Pat. No. 5,223,409), spores (U.S. Pat. No. 5,223,409), plasmids (Cull et al., 1992, PNAS USA 89:1865-1869), or phages (Scott and Smith 1990, Science 249:386-390; Devlin, 1990, Science 249:404-406; Cwirla et al., 1990, PNAS USA 87:6378-6382; Felici 1991, J. Mol. Biol. 222:301-310; and U.S. Pat. No.5,223,409).

To identify an RNR inhibitor, one can incubate a candidate compound with a cell-free preparation described above. One then evaluates the binding between the compound and the polypeptide, or the RNR activity of the cell-free system compared with that in the absence of the compound. Methods of evaluating RNR activity are well known in the art. If the level of the RNR activity is lower in the presence of the compound than that in its absence, the compound is identified as being useful for preventing and treating a cell proliferation-associated disorder.

As mentioned above, an RNR complex having a mutant subunit lacking one of the essential tyrosines loses the RNR activity substantially. Accordingly, a polypeptide of this invention that lacks one of the essential tyrosines can behave in a dominant negative manner to inhibit the RNR activity. Thus, such a polypeptide or a nucleic acid encoding it can be used for preventing and treating a cell proliferation-associated disorder

An RNA of this invention can also be used to inhibit the RNR activity in a cell, thereby preventing and treating a cell proliferation-associated disorder. The RNA inhibits the expression of an RNR subunit gene via RAN interference. RNA interference (RNAi) is a process in which double-stranded RNA (dsRNA) directs homologous sequence-specific degradation of messenger RNA. In mammalian cells, RNAi can be triggered by 21-nucleotide duplexes of small interfering RNA (siRNA) without activating the host interferon response. As this process represses the expression of an RNR subunit gene in the cells, it can be used to treat a cell proliferation-associated disorder.

An RNA of this invention can be synthesized by techniques well known in the art. See, e.g., Caruthers et al., 1992, Methods in Enzymology 211, 3-19, Wincott et al., 1995, Nucleic Acids Res. 23, 2677-2684, Wincott et al., 1997, Methods Mol. Bio. 74, 59, Brennan et al., 1998, Biotechnol Bioeng., 61, 33-45, and Brennan, U.S. Pat. No. 6,001,311. The RNA can also be transcribed from an expression vector and isolated using standard techniques. An RNA or vector of this invention can be delivered to target cells using method also well known in the art. See, e.g., Akhtar et al., 1992, Trends Cell Bio. 2, 139. For example, it can be introduced into cells using liposomes, hydrogels, cyclodextrins, biodegradable nanocapsules, or bioadhesive microspheres. Alternatively, the RNA or vector is locally delivered by direct injection or by use of an infusion pump. Other approaches include the use of various transport and carrier systems, for example though the use of conjugates and biodegradable polymers.

Also within the scope of this invention is a pharmaceutical composition that contains (1) an active agent, e.g., one of the above-descried antibodies, RNR mutant subunits, RNAi agents, or compounds identified to be RNR inhibitors; and (2) a pharmaceutically acceptable carrier and. Other active agents that can be used include an antisense oligonucleotide and a peptide or polypeptide containing an amino acid sequence at the C-terminus of RRM2. For example, a 7-amino acid peptide (FTLDADF) at the C-terminus of RRM2 was found to dramatically suppress the human RNR activity when added to the enzyme in vitro at a ratio of 10:1 (peptide:enzyme). It was further observed that a truncated form of the M2 subunit, lacking these 7 amino acids at the C-terminus, fails to form a complex with the corresponding large subunit M1. These results suggest that the 7-amino acid peptide competes with M2 for a binding site on M1. Such peptides or polypeptides can be generated and used according to the methods described above or any other methods well known in the art. The efficacy of the pharmaceutical composition for treating a cell proliferation-associated disorder can be preliminarily evaluated in vitro. For in vivo studies, the composition can be injected into an animal and its therapeutic effects are then accessed.

An active agent can be formulated into dosage forms for different administration routes utilizing conventional methods. For example, it can be formulated in a capsule, a gel seal, or a tablet for oral administration. Generally, an active agent will be suspended in a pharmaceutically-acceptable carrier (e.g., physiological saline) and administered orally or by intravenous infusion, or injected or implanted subcutaneously, intramuscularly, intrathecally, intraperitoneally, intrarectally, intravaginally, intranasally, intragastrically, intratracheally, or intrapulmonarily. For prevention and treatment of cancer, the compound can be delivered directly to the cancer tissue.

The dosage required depends on the choice of the route of administration; the nature of the formulation; the nature of the subject's illness; the subject's size, weight, surface area, age, and sex; other active agents being administered; and the judgment of the attending physician. Suitable dosages are in the range of 0.01-100.0 mg/kg. Wide variations in the needed dosage are to be expected in view of the variety of compounds available and the different efficiencies of various routes of administration. For example, oral administration would be expected to require higher dosages than administration by intravenous injection. Variations in these dosage levels can be adjusted using standard empirical routines for optimization as is well understood in the art. Encapsulation of the compound in a suitable delivery vehicle (e.g., polymeric microparticles or implantable devices) may increase the efficiency of delivery, particularly for oral delivery.

This invention further features a method for treating a cell proliferation-associated disorder. Subjects to be treated can be identified by standard methods for diagnosing such a disorder, or by determining an RNR subunit genomic DNA level, RNA level, protein level, or an RNR activity in a sample prepared from a subject. For example, if the level is higher in the sample from the subject than that in a sample from a normal subject, the subject is a candidate for treatment with an effective amount of compound that decreases the RNR activity. The term “treating” refers to administration of a composition to a subject, who has a cell proliferation-associated disorder, with the purpose to cure, alleviate, relieve, remedy, prevent, or ameliorate the disorder, the symptom of the disorder, the disease state secondary to the disorder, or the predisposition toward the disorder. An “effective amount” is an amount of the composition that is capable of producing a medically desirable result, e.g., as described above, in a treated subject. This method can be performed alone or in conjunction with other drugs or therapy.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications recited herein are hereby incorporated by reference in their entirety.

### EXAMPLE 1

In this example, Human RNR subunits were used to reconstitute active RNR. Human RNR has two homologous small subunits, hRRM2 and p53R2. Subunit p53R2 is directly regulated by the tumor suppressor p53 protein. It is believed that p53 plays a crucial role in G1 and G2 phase of the cell cycle, apoptosis, and regulating the supply of nucleotides in repairing damaged DNA (Tanaka, H. et al., 2000, Nature 404, 42-9; Levine, 1997, Cell 88, 323-31; and Guittet, et al., 2001, J. Biol. Chem. 276, 40647-51). It has been proposed that p53R2 interacts with p53 to form a protein-protein complex (Xue, et al., 2003, Cancer Res 63, 980-6). In response to genomic stress, p53 releases p53R2 and induces its transcription (Zhou et al., 2003, Cancer Res. 63, 6583-94). Free p53R2 binds to RI to prompt DNA repair (Guittet, et al., 2001, J. Biol. Chem. 276, 40647-51). Studies of enzymatic properties in response to iron chelators, radical quenchers, and other RNR inhibitors revealed differences in characteristics between p53R2 and hRRM2 (Shao et al., 2004, Cancer Res 64, 1-6).

To reconstitute an active RNR, three 6′His-tag human RNR subunit proteins (hRRM2, p53R2, and hRRM1) were prokaryotically expressed, purified using Ni-resin affinity chromatography, and stored in 50 mM Tris-HCl, pH 7.4, 100 mM KCl at −70° C. according to standard techniques. The purified proteins were subjected to SDS-PAGE analysis. It was found that the purified proteins were mostly intact. To reconstitute RNR, 1 μM of hRRM2 or p53R2 protein was incubated with 0.5 μM hRRM1 protein in multiple-well plates. The reconstitute RNR was tested for RNR activity according to standard techniques. It was found it possessed RNR activity.

The above described reconstitute RNR was used to screen for RNR inhibitors. Hydroxyurea (HU, a known RNR inhibitor), semicarbazide (SC), and 4 HU derivatives: schiff bases of hydroxysemicarbazide (“SB-HSC”) 2, 21, 24, and 29 were tested.

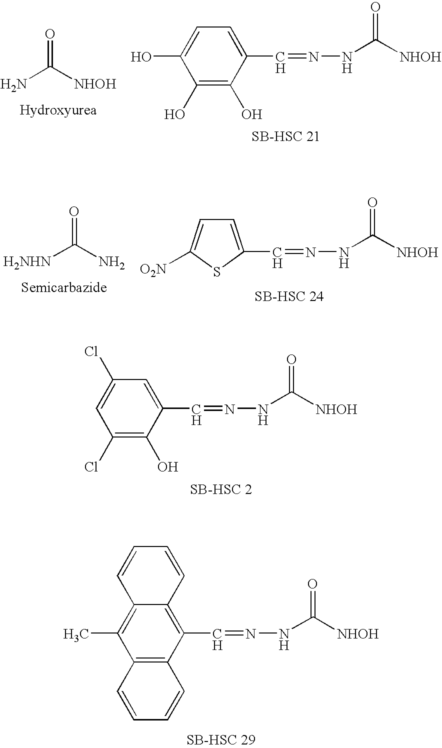

Each compound of various concentration was mixed with the purified hRRM2 and hRRM1, or p53R2 and hRRM1 at room temperature for 30 minutes. An enzymatic reaction buffer was then added to the mixture to reach a final volume of 100 μl and incubated at 37° C. for 30 minutes. The reaction buffer contained 0.125 mM [³H] CDP, 50 mM HEPES pH 7.2,6 mM DTT, 4 mM MgOAC, 2 mM ATP, 0.05 mM CDP, 100 mM KCl, and 0.24 mM NADPH. 60 mM FeCl₃ could be added to the buffer as an iron source. After dephosphorylation, the supernatant of each reaction mixture was analyzed by an HPLC-connected radioactivity detector. IC₅₀ values were calculated by the non-linear regression equations: f (x)=(a-d)/[1+(x/c)^{b}]+d.

The results show that HU significantly inhibited the activity of RNR containing hRRM2 or p53R2, indicating the reconstituted RNR can be used to screen for RNR inhibitors. It was found that HU inhibited the RNR activity in both cases more potently than SC. In the presence of irons and reductants, it inhibited hRRM2-containing RNR more potently than p53R2-containing-RNR. It was also found that DMSO inhibited p53R2-containing-RNR more potently than hRRM2-containing RNR, indicating DMSO is more specific for p53R2 than hRRM2. Interestingly, 1% and 2.5% DMSO had similar effects on HU's inhibition of both small subunits.

The above-described four SB-HSCs were tested for their inhibitory potency and subunit-selectivity by the in vitro RNR assay in the same manner descried above. The dose-dependent inhibition curves were found. The corresponding IC50 values are summarized in Table 1 below.

TABLE 1
IC50 values of RNR Inhibitors

| | In vitro RR activity (IC50 ± sd, uM) | | | | Cell-based assay |
|---|---|---|---|---|---|
| Compound | M2/M1 | p53R2/M1 | Average | Selective index | (IC50 ± sd, uM) |
| SC (in dH2O) | >5000 | >5000 | >5000 | NA | >2192 |
| HU (in dH2O) | 164.68 ± 7.95 | 190.37 ± 6.43 | 177.52 | 0.87 | 82.0 ± 6.0 |
| HU (in dH2O + FeCl3) | 555.70 ± 24.10 | 1986.16 ± 71.18 | 1270.93 | 0.28 | |
| HU (in 1% DMSO) | 147.57 ± 7.34 | 158.74 ± 6.82 | 153.16 | 0.93 | |
| HU (in 2.5% DMSO) | 155.17 ± 6.59 | 145.81 ± 6.06 | 150.49 | 1.06 | |
| SB-HSC21 (in 1% DMSO) | 11.48 ± 0.63 | 13.56 ± 0.70 | 12.52 | 0.85 | 2.7 ± 0.6 |
| SB-HSC24 (in 2.5% DMSO) | 51.99 ± 2.03 | 11.48 ± 0.60 | 31.73 | 4.53 | 10.6 ± 0.3 |
| SB-HSC2 (in 2.5% DMSO) | 110.73 ± 6.64 | 30.60 ± 1.62 | 70.66 | 3.62 | 6.5 ± 0.6 |
| SB-HSC29 (in 2.5% DMSO) | >250 | >250 | >250 | NA | 4.4 ± 0.0 |

The results indicate that all 4 SB-HSCs are potent RNR inhibitors with different specificities for p53R2-containing RNR more potently than hRRM2-containing RNR. The results were further corroborated by a standard cell-based assay and found to be comparable.

The results indicate that HU inhibited the RNR activity of both small subunits in a dose-dependent manner and the (N)—OH group was essential for the inactivation. In the presence of iron and reductants, HU had more favorable inhibition on hRRM2. It is also indicated that SB-HSC 29 had different mechanisms than other SB-HSCs.

Electron paramagnetic resonance (EPR) spectroscopy was then conducted. Prior to EPR, protein samples were incubated with each of the above-described compounds at room temperature for 30 minutes. EPR parameters were set as following: T=20 K, microwave frequency=9.376 GHz, microwave power=0.5 mW, modulation amplitude 4 gauss, modulation frequency 100 KHz. EPR measurement results showed that HU and four SB-HSCs quenched the tyrosyl radicals of both RNR small subunits. The observed trend in the tyrosyl radical EPR spectrum intensity was consistent with the changing RNR activity after treatment by these compounds. The EPR analysis was conduced in the presence of irons and the reductants. It was found that the tyrosyl radicals of both RNR small subunits were less quenched by HU in particular. This effect is more obvious on p53R2. Shown below is a scheme of the regeneration of the active-essential iron-tyrosyl radical center and hence, the RNR activity of both small subunits by incubation with iron and reductants after HU treatment.

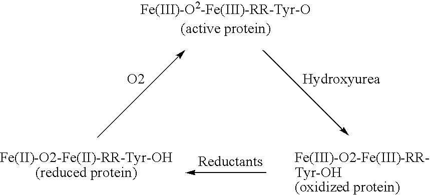

The results suggest that the above-described compounds inhibit the RNR activities in a dose-dependent manner and the (N)—OH group was essential for the inactivation. The inhibitory potency on RNR in order was SB-HSC 21, 24, 2, HU, and SB-HSC 29. SB-HSC 21 had almost the same inhibitory activities on both small subunits, whereas SB-HSC 2 and 24 were significantly more efficient in inactivating p53R2.

In summary, by reconstituting of recombinant subunit proteins (hRRM2 or p53R2 with hRRM1), a standard in vitro RNR assay was developed for high-throughput identification of novel RNR inhibitors for their potency and small subunit-selectivity. The above described assays can be conducted in parallel. For example, at least 100 samples can be analyzed every 24 hours by one set of detectors. Thus, they are suitable for high throughput screening. The study on the inhibitory mechanism can provide information necessary for rational design of novel RNR inhibitors.

### EXAMPLE 2

In this example in hRRM2 or p53R2 was identified to play a pivotal role in radical generation. Sequence alignment analysis of six Class I small RNR subunits were performed. These six RNR subunits were E.coliR2, ChlamydiaR2, YeastR2, MouseR2, hRRM2 and p53R2.

| | |
|---|---|
| | 112 127 134 317 |
| | \| \| \| \| |
| ChlamydiaR2 | IFKHVTNPEA RQYLLRQAFE EAVHTHTFLY ICESLGLD-- ... ... KNFFETRVIE YQHAASLTW |
| | 108 122 129 356 |
| | \| \| \| \| |
| E.coliR2 | LLPLISIPEL ETWVETWAFS ETIHSRSYTH IIRNIVNDPS ... ... VAPQEVEVSS YLVGQIDSEV |
| | 169 183 190 376 |
| | \| \| \| \| |
| YeastR2 | FSTEVQIPEA KSFYGFQIMI ENIHSETYSL LIDTYIKDPK ... ... TNFFEKRVSD YQKAGVMSKS |
| | 163 177 184 370 |
| | \| \| \| \| |
| MouseR2 | FSQEVQVTEA RCFYGFQIAM ENIHSEMYSL LIDTYIKDPK ... ... TNFFEKRVGE YQRMGVMSNS |
| | 162 176 183 369 |
| | \| \| \| \| |
| hRRM2 | FSQEVQITEA RCFYGFQIAM ENIHSEMYSL LIDTYIKDPK ... ... TNFFEKRVGE YQRMGVMSSP |
| | 124 138 145 331 |
| | \| \| \| \| |
| p53R2 | FSQEVQVPEA RCFYGFQILI ENVHSEMYSL LIDTYIRDPK ... ... TNFFEKRVSE YQRFAVMAET |

It has known that there are eight conserved tyrosines in hRRM2 and p53R2. Site-direct mutagenesis was used to replace these tyrosines with phenylalanines (F) or tryptophans (W). Mutation of Tyr162 to Phe162 in hRRM2, or Tyr124 to Phe124 in p53R2 was carried out by PCR using pET-hRRM2 or pET-p53R2 as a template. The oligonucleotides for hRRM2 Y162F and p53R2 Y124F were respectively 5′-ATTACAGAAGCCCGCTGTTTCTTTGGCTTCCAAATTGCCATGGAA-3′, and 5′GTTCCAGAGGCTCGCTGTTTCTTTGGCTTTCAAATTCTCATCGAG-3′ (boldface letters denote the mutated codon). Mutagenesis oligonucleotides were synthesized by an Applied Biosystems DNA/RNA synthesizer (Model 392).

PCR was conducted by pre-heating a mixture containing 1 ng of pET-hRRM2 or pET-p53R2,330 ng of each of the above-listed primers, and 0.2 mM dNTPs (Amersham Pharmacia Biotech) in a buffer recommended for Pfu polymerase (Stratagene) to 95° C. for 1 minute; then followed by 18 cycles of incubation at 95° C. for 30 seconds, 55° C. for 30 seconds, and 68° C. for 12 minutes. After being cooled to room temperature, one microliter of Dpn1 restriction enzyme was added to the reaction mixture. The mixture was then incubated at 37° C. for 1 hour. The resulting constructs containing mutant coding sequences were transfected into the E. coli strain BL21(DE3) (Stratagene, La Jolla, Calif.), and induced to express mutated hRRM2 and p53R2 polypeptides (“Y162F-M2” and “Y124F-p53R2,” respectively) by isopropyl-1-thio-β-D-galactopyranoside (IPTG).

More specifically, a 20 ml culture of transformed bacteria grown overnight was added to 1 L of LB medium containing 30 μg/ml kanamycin and grown at 37° C. for 2-3 hours (check O.D₆₀₀ not excessive 0.9). Protein expression was induced by 1 mM IPTG and bacterial growth continued for 16 hours at 30° C. Harvested cell pellets were disrupted by vigorous agitation and incubation with Bugburster and Benonase (Qiagen) at 4° C. for 30 minutes. Immobilized Ni(II) affinity chromatography resin (Ni-NTA, Qiagen) was suspended in the lysate supernatant at 4° C. for 30 minutes, then the resin was loaded on a gravity column and washed with at least 30 bed volumes of a buffer containing 50 mM NaH₂PO₄, 800 mM NaCl, 50 mM imidazole, 0.1% Triton-X 100, and 10 mM 2-Mercaptoethanol (pH 7.0). The protein was eluted with 50 mM NaH₂PO₄, 300 mM NaCl, and 125 mM imidazole (pH 7.0) and were dialyzed overnight at 4° C. against 2000× sample volumes of 25 mM Tris-HCl (pH 7.4). Expression and purification of hRRM1 was conducted in a similar way except that this protein was eluted without 2-Mercaptoethanol. Proteins thus-prepared were quantified by a protein assay kit (Bio-Rad, Hercules, Calif.). Their purities were determined by densitometric scanning of the coomassie-stained SDS-PAGE gels.

All mutants constructs (pET-hRRM2Y176F, pET-hRRM2Y162F, pET-p53R2Y138F, pET-p53R2Y124F, pET-hRRM2Y369F, pET-p53R2Y331F, pET-hRRM2Y176W, pET-hRRM2Y162W, pET-p53R2Y138W, and pET-p53R2Y124W) and respective mutant proteins were produced in the same manner described above.

X-band Electron Paramagnetic Resonance (EPR) Spectra were measured with a Bruker EMX spectrometer equipped with an Oxford helium cryostat. Purified hRRM2 and p53R2 proteins were frozen in liquid nitrogen prior to insertion in the cavity. Instrumental parameters were T=20 K, microwave frequency=9.376 GHz, microwave power=0.5 mW, modulation amplitude 4 gauss, modulation frequency 100 KHz. It was found that the 9 GHz EPR spectra of wild-type E. coli, mouse, and human RNR small subunits were similar, indicating that in all three enzymes the dihedral angle between the tyrosine Cβ-H bond and the axis perpendicular to the aromatic ring are close (Pesavento et al., 2001, Adv Protein Chem 58, 317-85. EPR measurements of wild-type hRRM2 and p53R2 yielded typical RNR protein tyrosyl radical signals. Mutants Y176F in hRRM2 and Y138F in p53R2 (“Y176F-M2” and “138F-p53R2,” respectively) resulted in EPR-silent proteins, indicating that Tyr176-M2 and Tyr138-p53R2 are residues hosting the stable radical in hRRM2 and p53R2, respectively. This is in accord with previous studies in E.coli and mouse. Unexpectedly, Y162F-M2 and Y124F-p53R2 mutants were EPR-silent as well. Mutations of either one of the two tyrosines (Tyr162-M2 and Tyr176-M2; Tyr124-p53R2 and Tyr138-p53R2) obliterated the stable tyrosyl radical, indicating that both of the tyrosine residues are crucial for the formation of the stable tyrosyl radical in the human RNR, and they function in a correlative manner. No significant changes were observed in EPR spectra of the other six tyrosine-mutants, signifying that these tyrosine residues are not essential for the radical formation.

The activities of hRRM2/hRRM1 and p53R2/hRRM1 were measured using a modified [³H] CDP reduction assay. The reaction mixture contained 0.125 μM [³H] CDP (0.3 μCi), 50 mM of HEPES (pH 7.2), 6 mM DTT, 4 mM MgOAC, 2 mM ATP, 0.05 mM CDP, 100 mM KCl, 0.24 mM NADPH. The enzyme activity was measured in the presence of 0.5 μM of M1 protein and 1 μM of hRRM2 or p53R2 proteins with a total sample volume of 100 μl. After incubation at 37° C. for 15-30 minutes and dephosphorylation with phosphodiesterase, the supernatant was analyzed by HPLC using a C-18 reversed phase column to separate [³H] cytidine and [³H] deoxycytidine. Radioactivity of the HPLC aliquots was measured with a β-RAM-2B Flow-through radioisotope beta/gamma detector (IN/US Systems, Tampa, Fla.). The enzyme activity was measured as relative activity of dCDP/(CDP+dCDP)% (i. e., the percentage of dCDP of all ribonucleoside diphosphates and deoxyribonucleoside diphosphates). Results were normalized against those of wild type proteins, which were considered having 100% conversion of CDP to dCDP. The specific enzyme activity was reported as nmol of dCDP formed/min/mg protein and summarized in Table 2 below. Each value is the average of two to three determinations with deviations <0.3%.

TABLE 2
RNR Activity of the Mutated R2 Proteins

| Mutation | p53R2 | Activity(%) | hRRM2 | Activity(%) |
|---|---|---|---|---|
| Y->F | 124 | ND | 162 | 3.6 |
| | 138 | ND | 176 | 2.4 |
| | 331 | ND | 369 | ND |
| Y->W | 124 | 27.8 | 162 | ND |
| | 138 | ND | 176 | ND |

Note:
ND = not detectable.

Consistent with the EPR results, no activities were observed for the Y138F and Y124F mutants of p53R2, confirming the importance of these two tyrosines. Also, minimal activity in Y176F (<2.5% of the native protein), as well as in Y162F (<3.6% of the native protein) of hRRM2 were detected. The remaining activities of these two mutants evoke great interest. The low activity found in the Y176F-M2 mutant suggests that the formation of other transient or stable radicals in hRRM2 is responsible for the catalysis of substrate reduction. This observation corresponds with previous studies in E. coli and mouse (Bollinger et al., 1991, Science 253, 292-8 and Sahlin et al., 1994, J. Biol. Chem. 269, 11699-702. The similar low activity found in the Y162F-M2 mutant indicates that (1) Tyr162-M2 is a site forming other radicals in the mutant Y176F-M2; (2) human RNR needs both Tyr176 and Tyr162 in its small subunit to be fully active. On the other hand, the Y369F-M2/Y331F-p53R2 mutant of human RNR demonstrates the typical EPR signal, yet has no detectable enzymatic activity. This tyrosine aligns with Tyr370 in mouse/Tyr356 in E. coli, which is a part of the long-range electron-transfer pathway at the R1-R2 interface (Climent et al., 1992, Biochemistry 31, 4801-7 and Rova et al., 1999, J. Biol. Chem. 274, 23746-51). Therefore, mutation of this residue inhibits radical transfer and hence enzymatic activity, but not the stable radical formation in small subunits. The results confirm that this tyrosine is part of the electron-transfer pathway of human RNRs as well.

Since there was no human R2 crystal structure available, three-dimensional (3D) homology models of hRRM2 and p53R2 were built based on a recently published X-ray crystal structure of murine p53R2 (PDB ID 1W69). The eight tyrosines mentioned above were mapped on the structures to elucidate their possible function roles. Sequence alignment indicated that hRRM2 and p53R2 share high homology (95.5% and 81.2% respectively) with the murine R2 within 288 amino acids ranging from residues Asn65 to Glu352 using structures in 1W69. The models were evaluated using the SYBYL Protable module to assure the accuracy of the protein structures. As shown in the X-ray structure of the reduced murine R2, the binuclear iron center is located in a four-helix bundle in p53R2 and hRRM2 (Strand et al., 2003, Biochemistry 42, 12223-34). The hRRM2 structure model, as well as p53R2, showed that each iron atom is coordinated by four ligands: His134, Asp100, Glu131 and Glu228 for Fe1; and His231, Glu194, Glu228, and Glu131 for Fe2. The two carboxylates, Glu131 and Glu228, bridge the two iron ions, which are separated by 3.4 Å. The two tyrosines are located at opposite sides of the dinuclear iron cluster, each close to one iron. The hydroxyl oxygen of Tyr176-M2 is 5.6 Å from Fe1 and the hydroxyl oxygen of Tyr162-M2 is 10.8 Å from Fe2. The model indicated that both tyrosines are enclosed in hydrophobic environments with remarkable similarity. However, Tyr176 is surrounded mostly by leucines and isoleucines, whereas Tyr162 is enclosed by phenylalanines.

Tyr162-M2/Tyr124-pS3R2, like Tyr176-M2/Tyr138-p53R2, is important for human RNR function. However, whether Tyr162/Tyr124 possesses a radical, transient or stable, was examined. EPR spectra simulation (Rova et al., 1999, J. Biol. Chem. 274, 23746-51) was conducted. The results demonstrated that Tyr162-M2 could harbor a radical with an EPR spectrum very similar to that of Tyr176-M2 (Svistunenko et al., 2004, Biophys. J. 87, 582-95). The method, established by Svistunenko and Cooper, predicts the EPR spectra of tyrosyl radical sites in a protein by comparing the rotational/dihedral angle (θ) of a tyrosine phenoxyl ring with the simulated value from the shape of the EPR spectra of tyrosyl radicals. Svistunenko and Cooper simulated the rotational angle of the stable tyrosyl radical (Tyr177) from the high field and X-band EPR spectra of the murine M2 subunit and calculated the dihedral angle of Tyr177 in a murine M2 crystal structure (PDB ID 1XSM, 96% homology with hRRM2 (Schmidt et al., 1996, J. Biol. Chem. 271, 23615-8; Sahlin et al., 1987, Biochemistry 26, 5541-8; and Kauppi et al., 1996, J. Mol. Biol. 262, 706-20). They found that the calculated value of the dihedral angle of radical holding tyrosines should be θ=3°. In this example, rotation angles of all the tyrosines available were calculated in 1×SM using the Svistunenko and Cooper method (Table 3).

TABLE 3
Rotational angles of the tyrosine residues in mouse R2 and hRRM2

| Tyrosine (Mouse) | Tyrosine (hRRM2) | Angle θ |
|---|---|---|
| 88 | 87 | 27.8 |
| 95 | 94 | 98.6 |
| 163 | 162 | 6.5 |
| 177 | 176 | 18.3 |
| 184 | 183 | 164.2 |
| 193 | 192 | 77.6 |
| 222 | 221 | 84.2 |
| 324 | 323 | 77.4 |

Note:
Bold = possible tyrosyl radical harboring residues.

As shown in Table 3, mouse Tyr177 and Tyr163 have rotational angles that agree well with the simulated result (θ=3°). The calculation of the rotational angle of Tyr177 (θ=18.3°) matches Svistunenko and Cooper's result. Yet, remarkably, the rotation angle of Tyr163 (θ=6.5°), in alignment with human Tyr162, was found closest to the simulated stable tyrosyl radical value (θ=3°). According to this method, a radical at Tyr163 should have a similar EPR spectrum as Tyr177. Comparisons between the structures reveal that human Tyr162 and murine Tyr163 are each surrounded by ten identical amino acid residues, which indicate that the calculations based on the murine structure can be applied to the human protein.

Calculation analysis indicates that both human Tyr162-M2 and Tyr176-M2 have similar dihedral angles, which demonstrate that the EPR signals of these two tyrosines may overlap. These calculation results are supported by the above-described site-directed mutagenesis observations in that human Tyr162-M2 can be a radical site.

A second mutagenesis study was conducted to further differentiate and compare the two-tyrosyl radicals. Each of human Y176 and Y162 was mutated to a tryptophan (W), another redox-active amino acid similar to tyrosine. It was found that either Y176W-M2 or Y162W-M2 mutants exhibited silent EPR signals, indicating that tryptophan substitutions cannot enable stable radical formation in hRRM2. This observation reinforces the idea that both of the two tyrosines are critical in the radical formation and that they are correlated. In contrast to phenylalanine mutants (Y176F and Y162F), tryptophan mutants (Y176W and Y162W) are completely enzymatically inactive (see Table 2 above). This difference is due to disparities in the radical harboring ability of phenylalanine and tryptophan. The radical formed by the two-amino-acid-correlated-radical-center decays faster with a tryptophan than with a non-radical phenylalanine substitution. This interpretation is supported by the observation in that the turnover of substrate per tryptophan mutant (Y177W in mouse) is less than that of phenylalanine mutant (Y177F in mouse) (Potsch, et al., 1999, J. Biol. Chem. 274, 17696-704). It was also found that Y138W-p53R2 resulted in EPR silent and enzymatically inactive proteins as well. However, Y124W-p53R2 retained 1/3 EPR signal content and enzyme activity of the wild type protein. A neighboring residue difference between hRRM2 and p53R2 may explain this incongruity.

The results from the EPR spectroscopy and the first mutagenesis study indicate that Tyr162-M2/Tyr124-p53R2 is critical, much like that of Tyr176-M2/Tyr138-p53R2, for the formation of the stable tyrosyl radical and enzymatic activity of human RNRs. In addition to the mutual need of the two tyrosines for radical formation and enzyme activity, the structural proximity of Tyr176 and Tyr162 to the diiron cluster increases the likelihood that Tyr176 and Tyr162, together with the dinuclear cluster, form a diferric-dityrosyl radical cofactor center in the human RNR.

In the second mutagenesis study noted above, the complete lack of enzyme activity in tryptophan mutants excludes the possibility that the remaining activities found in Y176F and Y162F phenylalanine mutants were due to any other intrinsic activities unrelated to the dityrosyl radical center, or due to a contamination with wild-type protein. All observed activities, however small, were real. Simulation calculation further suggest that Tyr162-M2/Tyr124-p53R2 is capable of harboring a radical. However, it is still intriguing that no additional tyrosyl radical signal was observed in EPR spectra of human RNR small subunits. This phenomenon could come from two reasons: (1) similar rotational angles (θ=6.5° for Tyr162-M2, θ=18.5° for Tyr176-M2) render the two tyrosyl radical EPR signals undistinguishable, or (2) Tyr162-M2/Tyr124-p53R2 gives a transient radical that decays before detection.

The above results indicate both similarities and differences between p53R2 and hRRM2. p53R2 is homologous to hRRM2 with a 83% sequence identity. In the first mutagenesis study noted above, both p53R2 and hRRM2 yielded EPR-silent proteins. Conversely, Y138F-p53R2 and Y124F-p53R2 mutants had no detectable enzyme activity, unlike Y176F-M2 and Y162F-M2 (Table 2). In the second mutagenesis study, similarities in both EPR spectra and activity were observed between the tryptophan substitutions Y138W-p53R2 and Y176W-M2 (Table 2). However, it is interesting that the Y124W-p53R2 mutant retained about one third of the radical content and activity of the native enzyme, whereas Y162W-M2 showed no EPR signal and enzymatic activity. The dissimilarities between p53R2 and hRRM2 could be attributed to a single amino acid difference between the two proteins near the cofactor center. The general conclusions in the formation of a dityrosyl radical center for hRRM2 are applicable to p53R2, despite differences in the two small subunits. The fact that Y124F-p53R2 is EPR-silent while Y124W-p53R2 is partially EPR-active indicates that a radical is harbored at Tyr124-p53R2 as suggested by the EPR simulation calculation.

As mentioned above, E. coli RNR has been used as the model organism for Class I RNR. It was believed that the radical generation of all Class I enzymes followed a similar route (Stubbe et al., 1998, Chem. Rev. 98, 705-762). However, as shown above, sequence alignment of various RNRs reveals that while the sequences of mammalian small subunits are very similar (80% homology), they are distant from Class I enzymes of other species (e.g. 25% homology with E. coli). The amino acid composition in proximity of Fe2 of the diferric cluster differs significantly between murine and E. coli R2 (Kauppi et al., 1996, J. Mol. Biol. 262, 706-20). In particular, E. coli R2 lacks a tyrosine at position 108, which corresponds to Tyr163 in mouse and Tyr162-M2/Tyr124-p53R2 in human near the Fe2 site. It is interesting to note that the transient formation of some tryptophan radicals in proximity of the Fe2 site has been observed in Y122F E. coli, giving 1-2% wild-type activity (Sahlin et al., 1994, J. Biol. Chem. 269, 11699-702; Sahlin et al., 1999, J. Biol. Chem. 274, 17696-704; Katterle et al., 1997, J. Biol. Chem. 272, 10414-21; and Larsson et al., 1986. Embo. J. 5, 2037-40). Another R2 subclass (Class Ic) was defined in Chlamydia RNR (Hogborn et al.,2004, Science 305, 245-8). It is intriguing that this class of R2 lacks the canonical tyrosyl radical site near Fe1, but an EPR signal with a shorter lifespan was detected. Structural alignment indicates that two tyrosine residues in vicinity of the Fe2 site (Tyr112 and Tyr189) in Chlamydia R2 could be candidates for an alternative tyrosyl radical. The study described herein suggests that Tyr162-M2/Tyr124-p53R2 in human and Tyr163 in mouse RNRs could be responsible for an alternative radical site. It appears that mammals have a more complex radical generating mechanism than that of bacteria.

The diiron cluster of murine R2 is found to be much more mobile and accessible than that of E. coli R2 (Strand et al., 2004, J. Biol. Chem. 279, 46794-801; Kauppi et al., 1996, J. Mol. Biol. 262, 706-20; and Strand et al., 2003, Biochemistry 42, 12223-34), favoring the formation of a dityrosyl radical cofactor center. Structural studies indicate while both tyrosines in the cofactor center are buried in the enzyme interior, Tyr162-M2/Tyr124-p53R2 sits closer to the surface than Tyr176-M2/Tyr138-p53R2 does, making the former more accessible. The high mobility and accessibility of the radical-iron center could make mammalian RNRs more efficient in radical generation, reactivity, and transportation (Strand et al., 2004, J. Biol. Chem. 279, 46794-801; and Atta et al., J. Am. Chem. Soc. 116, 6429-6430). In addition, binding of ferrous iron in murine R2 is highly cooperative in contrast with that of E.coli R2 (Strand et al., 2003, Biochemistry 42, 12223-34), echoing the highly cooperative characteristic of the two tyrosines in mammal R2. Furthermore, the additional tyrosyl radical could supply the fourth electron in the radical reconstitution reaction, where four electrons are required for dioxygen reduction and only three are known to be provided by the protein, in which two are from ferrous ions and one from the known tyrosyl radical (Bollinger et al., 1991, Science 253, 292-8).

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claim.

1. An isolated polypeptide comprising an immunogenic fragment of SEQ ID NO: 1 or 15, wherein the fragment is at least 10 amino acid residues in length and spanning residue Y162 or Y369 of SEQ ID NO: 1, or residue Y124 or Y331 of SEQ ID NO: 15.

2. The polypeptide of claim 1, wherein the immunogenic fragment contains SEQ ID NO: 31 or 33.

3. The polypeptide of claim 2, wherein the immunogenic fragment contains SEQ ID NO: 35 or 37.

4. An isolated polypeptide comprising an mutant fragment of SEQ ID NO: 1 or 15, wherein the mutant fragment is at least 10 amino acid residues in length and is identical to a wild type fragment spanning the Y162 or Y369 residue of SEQ ID NO: 1, or spanning the Y124 or Y331 residue of SEQ ID NO: 15, except that the residue at position 162 or 369 of SEQ ID NO: 1 or at position 124 or 331 of SEQ ID NO: 15 is a non-tyrosine residue.

5. The polypeptide of claim 4, wherein the non-tyrosine residue is a phenylalanine or a tryptophan.

6. The polypeptide of claim 4, wherein the wild type fragment contains SEQ ID NO: 31 or 33.

7. The polypeptide of claim 6, wherein the wild type fragment contains SEQ ID NO: 35, 37, 39, or 41.

8. The polypeptide of claim 7, wherein the mutant fragment contains SEQ ID NO: 3, 5, 7, 9, 11, 13, 17, 19, 21, 23, 25, or 27.

9. An isolated nucleic acid comprising a sequence encoding the polypeptide of claim 1, or a complement thereof.

10. The nucleic acid of claim 9, wherein the sequence contains SEQ ID NO: 32 or 34.

11. The nucleic acid of claim 10, wherein the sequence contains SEQ ID NO: 36, 38, 40, or 42.

12. A vector comprising the nucleic acid of claim 9.

13. A host cell comprising the nucleic acid of claim 9.

14. A method of producing a polypeptide, comprising culturing the host cell of claim 13 in a medium under conditions permitting expression of a polypeptide encoded by the nucleic acid, and purifying the polypeptide from the cultured cell or the medium of the cell.

15. An isolated nucleic acid comprising a sequence encoding a polypeptide containing an mutant fragment of SEQ ID NO: 1 or 15, or a complement thereof, wherein the mutant fragment is at least 10 amino acid residues in length and is identical to a wild type fragment spanning the Y162 or Y369 residue of SEQ ID NO: 1 or spanning the Y124 or Y331 residue of SEQ ID NO: 15, except that the residue at position 162 or 369 of SEQ ID NO: 1 or at position 124 or 331 of SEQ ID NO: 15 is a non-tyrosine residue.

16. The nucleic acid of claim 15, wherein the wild type fragment of SEQ ID NO: 1 or 15 contains SEQ ID NO: 31 or 33.

17. The nucleic acid of claim 16, wherein the wild type fragment of SEQ ID NO: 1 or 15 contains SEQ ID NO: 35, 37, 39, or 41.

18. A vector comprising the nucleic acid of claim 15.

19. A host cell comprising the nucleic acid of claim 15.

20. A method of producing a polypeptide, comprising culturing the host cell of claim 19 in a medium under conditions permitting expression of a polypeptide encoded by the nucleic acid, and purifying the polypeptide from the cultured cell or the medium of the cell.

21. An RNA for inhibiting expression of a gene encoding SEQ ID NO: 1 or 15, the RNA comprising a first nucleotide sequence that hybridizes under stringent conditions to a segment of the gene, and a second nucleotide sequence that is complementary to the first nucleotide sequence and hybridizes to the first nucleotide sequence to form a duplex structure.

22. The RNA of claim 21, wherein the first nucleotide sequence and the second nucleotide sequence are on the same strand.

23. The RNA of claim 21, wherein the RNA is a double-stranded RNA.

24. The RNA of claim 21, wherein the first nucleotide sequence is at least 19 nucleotides in length.

25. The RNA of claim 24, wherein the first nucleotide sequence is 19 to 29 nucleotides in length.

26. A DNA vector comprising a nucleic acid that encodes the RNA of claim 21.

27. A method of identifying a compound for inhibiting the enzymatic activity of a ribonucleotide reductase, the method comprising
contacting a compound with a polypeptide of claim 1; and
determining binding, if any, between the compound and the polypeptide,
wherein a presence of the binding indicates that the compound is a candidate for inhibiting the enzymatic activity of a ribonucleotide reductase.

28. The method of claim 27, wherein the compound is a small organic molecule, a small inorganic molecule, an oligonucleotide, a peptide, a protein, or a carbohydrate.

29. A reconstituted dimeric ribonucleotide reductase having a ribonucleotide reductase activity, comprising:
a first purified polypeptide containing the sequence of a first subunit of a naturally occurring ribonucleotide reductase; and
a second purified polypeptide containing the sequence of a second subunit of the naturally occurring ribonucleotide reductase.

30. The preparation of claim 29, wherein the ribonucleotide reductase is a human ribonucleotide reductase.

31. The preparation of claim 30, wherein the first subunit is an R1 subunit.

32. The preparation of claim 31, wherein the second subunit is an R2 subunit or a p53R2 subunit.

33. A method of identifying a compound for treating a cell proliferation-associated disorder, the method comprising:
incubating a compound with a reconstituted dimeric ribonucleotide reductase of claim 27, and
determining a level of ribonucleotide reductase activity of the ribonucleotide reductase,
wherein the compound is determined to be effective in treating the cell proliferation-associated disorder if the level of the ribonucleotide reductase activity is lower than that determined in the same manner except that the compound is absent.

34. The method of claim 33, wherein the compound is a small organic molecule, a small inorganic molecule, an oligonucleotide, a peptide, a protein, or a carbohydrate.
